(19) United States
(12) Patent Application Publication
(10) Pub. No.: US 2026/0260356 A1
(43) Pub. Date: Sep. 3, 2026

(54) SYSTEM AND METHODS FOR IDENTIFYING INFLAMMATION BIOMARKERS IN RETINAL SCANS

(71) Applicant: Bonafia Inc., Palo Alto, CA (US)

(72) Inventors: Jan Rydfors, Palo Alto, CA (US); Jochen Kumm, Palo Alto, CA (US)

(21) Appl. No.: 19/555,687

(22) Filed: Mar. 3, 2026

Related U.S. Application Data
(60) Provisional application No. 63/766,291, filed on Mar. 3, 2025.
(60) Provisional application No. 63/880,133, filed on Sep. 11, 2025.

(51) Int. Cl. G06T 7/00; A61B 3/00; A61B 3/12; G06T 7/11; G16H 10/20; G16H 10/60

(52) U.S. Cl. CPC G06T 7/0016; A61B 3/0083; A61B 3/12; G06T 7/11; G16H 10/20; G16H 10/60; G06T 2207/20021; G06T 2207/20081; G06T 2207/30041

(57) ABSTRACT

One variation of the method includes: accessing a retinal scan; segmenting a layer of the first retinal scan into a set of regions; for each region in the set of regions: extracting a constellation of inflammation biomarkers from the region; calculating an intra-region metric representing spatial patterns between inflammation biomarkers in the constellation of inflammation biomarkers; and calculating an inter-region metric representing relationships between the constellation of inflammation biomarkers and intra-region metrics of tiles in the set of regions. The method also includes: calculating an inter-layer metric, representing systemic persistence of inflammation-related biomarker spatial patterns, based on inter-region metrics for each layer of the retinal scan; accessing an inflammation function relating inflammation biomarkers with an inflammation metric indicative of inflammation-related states; calculating an inflammation metric value based on the inter-layer metric and the inflammation function; and generating an inflammation-related state indication based on the inflammation metric value.

### CROSS-REFERENCE TO RELATED APPLICATIONS

This Application claims priority to U.S. Provisional Application No. 63/880,133, filed on 11 Sep. 2025, and U.S. Provisional Application No. 63/766,291, filed on 3 Mar. 2025, each of which is incorporated in its entirety by this reference.

### TECHNICAL FIELD

This invention relates generally to the field of imaging diagnostics and, more specifically, to a new and useful method for identifying inflammation-related state indicators from inflammation biomarkers detected in retinal scans in the field of imaging diagnostics.

### BRIEF DESCRIPTION OF THE FIGURES

FIG. 1 is a schematic representation of a method;

FIG. 2 is a flowchart representation of one variation of the method;

FIGS. 3A and 3B are flowchart representations of one variation of the method;

FIG. 4 is a flowchart representation of one variation of the method;

FIG. 5 is a flowchart representation of one variation of the method; and

FIG. 6 is a schematic representation of a system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

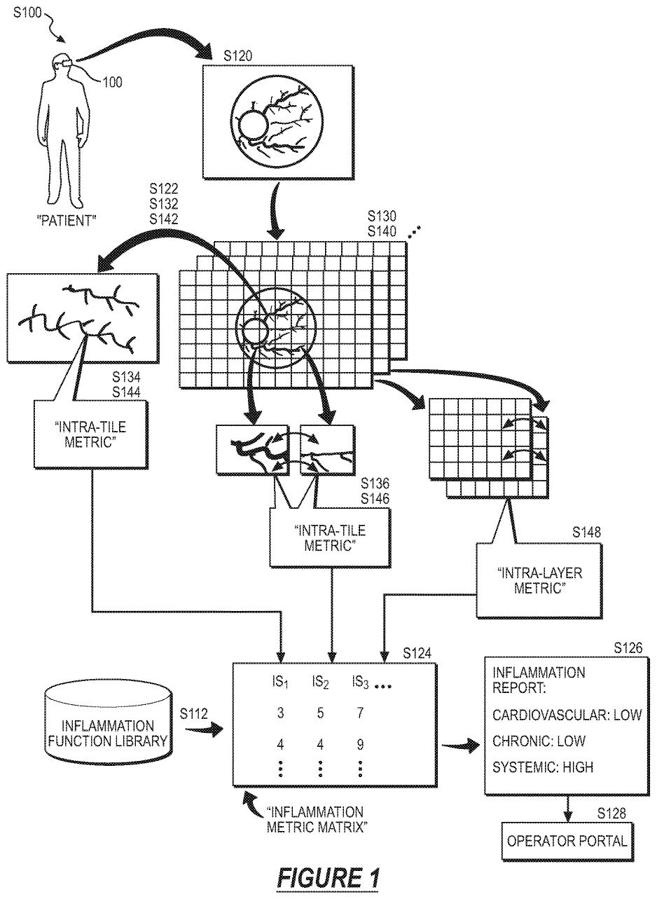

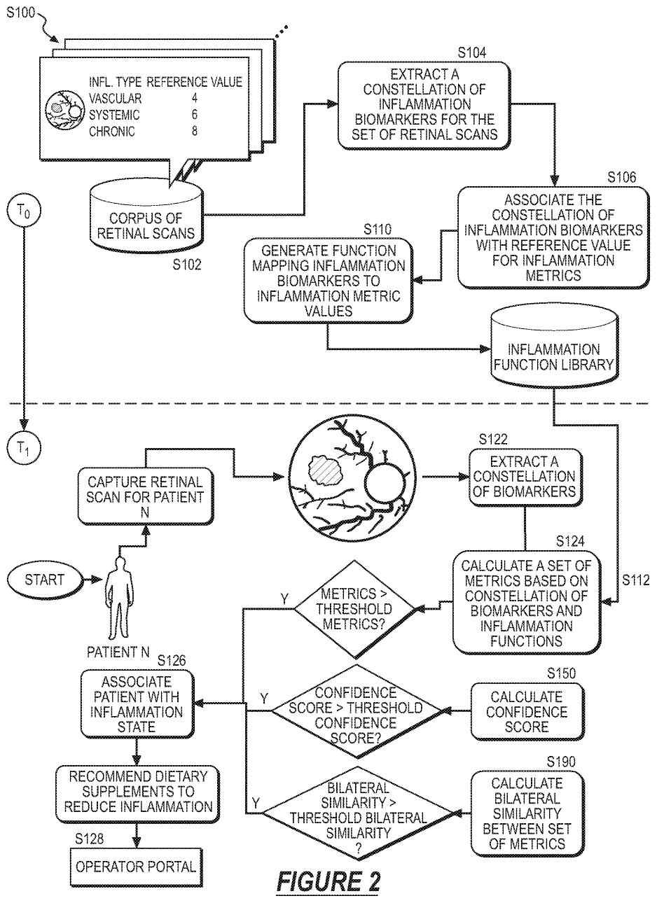

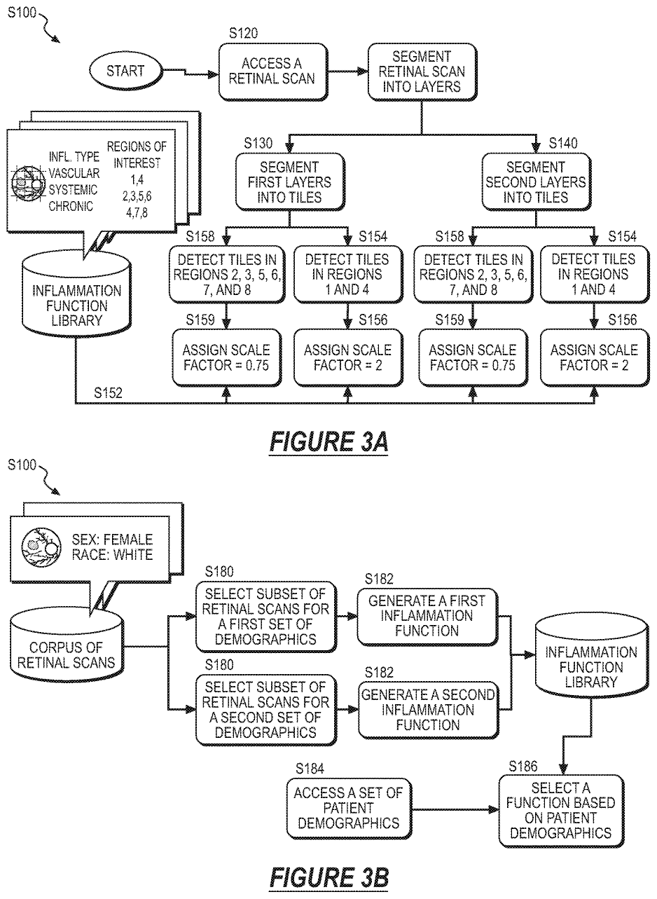

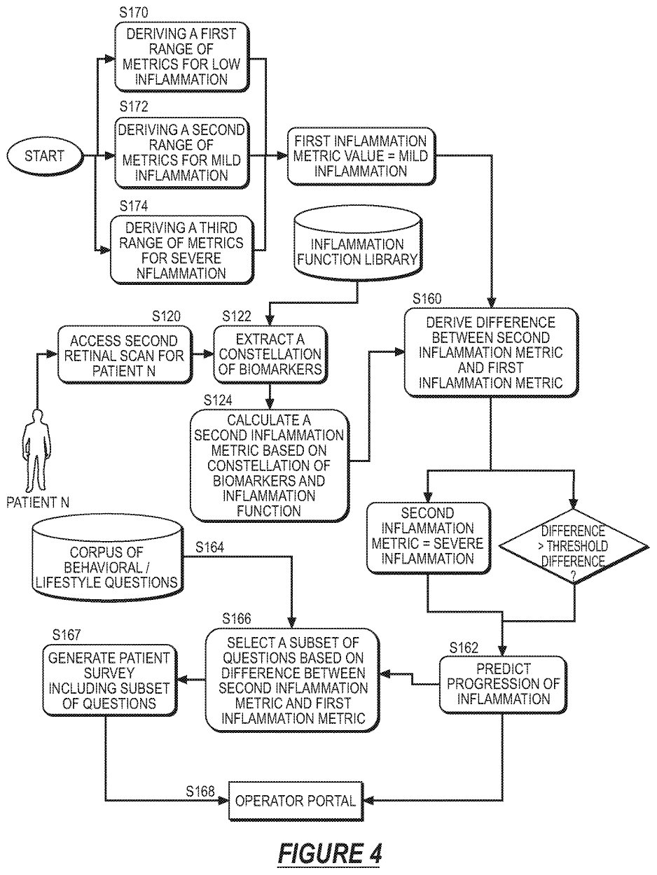

As shown in FIGS. 1-4, a method S100 includes, during a training time period, accessing a set of historical retinal scans, each historical retinal scan in the set of historical retinal scans tagged with a reference value of a first inflammation metric representing an inflammation-related state in Block S102.

The method S100 also includes, for each historical retinal scan in the set of historical retinal scans: extracting a first constellation of inflammation biomarkers in Block S104; and associating the first constellation of inflammation biomarkers with the first inflammation metric in Block S106.

The method S100 further includes generating a first inflammation function relating inflammation biomarkers with the first inflammation metric based on associations between constellations of inflammation biomarkers and a corresponding reference value of the first inflammation metric in Block S110.

The method S100 also includes, during an execution time period succeeding the training period: accessing a first retinal scan, captured by a retinal scanning system, for a first patient in Block S120; extracting a second constellation of inflammation biomarkers from the first retinal scan in Block S122; and calculating a first inflammation metric value, for the first inflammation metric, based on the first inflammation function and the second constellation of inflammation biomarkers in Block S124.

The method S100 further includes, in response to the first inflammation metric value exceeding a threshold inflammation metric value: generating an inflammation-related state indication corresponding to the first inflammation-related state for the first patient in Block S126; and serving the first inflammation metric value and the inflammation-related state indication to an operator portal in Block S128.

### 1.1 Variation: Segmenting

In one variation, the method S100 includes: accessing a first retinal scan, captured by a retinal scanning system, for a first patient in Block S120; and segmenting a first layer of the first retinal scan into a first array of tiles (and/or “regions”, “tokens”) of a first tile density in Block S130.

This variation of the method S100 also includes, for each tile in the first array of tiles: extracting a constellation of inflammation biomarkers from the tile in Block S132; and calculating an intra-tile metric, in a first set of intra-tile metrics, representing spatial patterns between inflammation biomarkers in the constellation of inflammation biomarkers in Block S134.

This variation of the method S100 further includes calculating an inter-tile metric, in a first set of inter-tile metrics, representing relationships between the constellation of inflammation biomarkers and intra-tile metrics of tiles in the first set of intra-tile metrics in Block S136; and segmenting a second layer of the first retinal scan into a second array of tiles of a second tile density less than the first tile density in Block S140.

This variation of the method S100 also includes, for each tile in the second array of tiles: extracting a constellation of inflammation biomarkers from the tile in Block S142; and calculating an intra-tile metric, in a second set of intra-tile metrics, representing spatial patterns between inflammation biomarkers in the constellation of inflammation biomarkers in Block S144.

This variation of the method S100 further includes calculating an inter-tile metric, in a second set of inter-tile metrics, representing relationships between the constellation of inflammation biomarkers and intra-tile metrics of tiles in the second set of intra-tile metrics in Block S146; calculating an inter-layer metric based on the first set of intra-tile metrics, the first set of inter-tile metrics, the second set of intra-tile metrics, and the second set of inter-tile metrics in Block S148; accessing a first inflammation function relating inflammation biomarkers with a first inflammation metric representing an inflammation-related state in Block S112; and calculating a first inflammation metric value based on the inter-layer metric and the first inflammation function in Block S124.

This variation of the method S100 further includes, in response to the first inflammation metric value exceeding a threshold inflammation metric value: generating an inflammation-related state indication corresponding to the first inflammation-related state for the first patient in Block S126; and serving the first inflammation metric value and the inflammation-related state indication to an operator portal in Block S128.

### 1.2 Variation: Inflammation Vectors

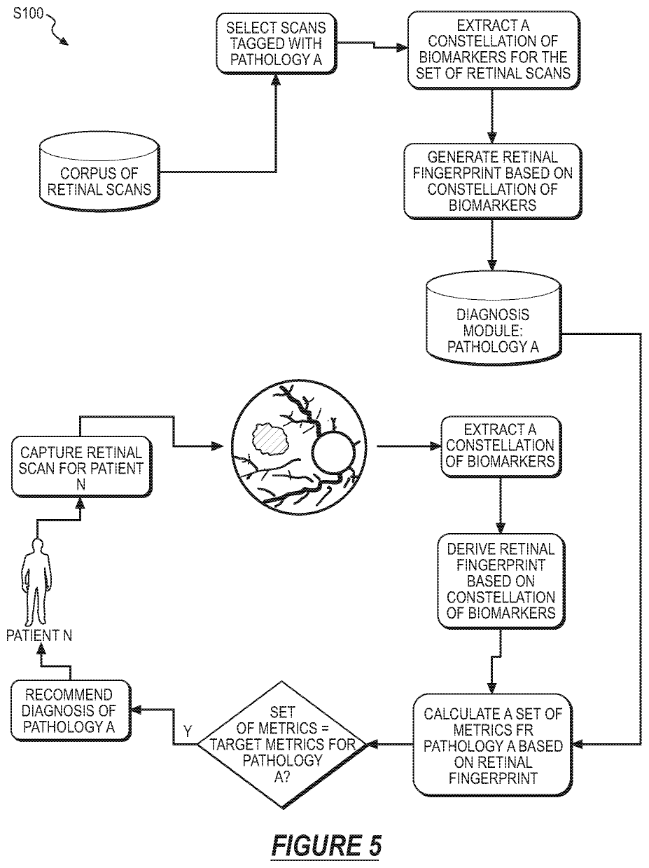

As shown in FIG. 5, one variation of the method S100 includes: capturing a first retinal scan for a first patient; extracting a first constellation of inflammation biomarkers from the first retinal scan; generating a first inflammation vector representing the first constellation of inflammation biomarkers; accessing a vector database representing a set of inflammation vectors; for each inflammation vector in the set of inflammation vectors, calculating a similarity score for the first biomarker; in response to a first similarity score, associated with a first inflammation vector, exceeding a threshold similarity score, associating the first patient with the first inflammation vector; generating a recommendation for diagnosis of the first patient with a first inflammation-related state associated with the first inflammation vector; and serving the recommendation to a medical operator.

### 1.3 Variation: Module Generation

One variation of the method S100 includes: receiving a corpus of retinal scans for a population of users, each retinal scan associated with an inflammation-related state; selecting a first set of retinal scans, in the corpus of retinal scans, characterized by a first inflammation-related state; for each retinal scan in the first set of retinal scans, extracting a first set of inflammation biomarkers from the retinal scan and generating an inflammation vector, in a set of inflammation vectors, based on the first set of inflammation biomarkers; generating a diagnostic module based on the set of inflammation vectors, the diagnostic module representing diagnostic indicators for the first inflammation-related state according to the set of inflammation vectors; and storing the diagnostic module in a vector database representing a corpus of inflammation-related states.

### 1.4 Variation: n-Dimensional Space Projection

One variation of the method S100 includes: capturing a first retinal scan for a first patient; extracting a first constellation of inflammation biomarkers from the first retinal scan; generating a first inflammation vector representing the first constellation of inflammation biomarkers; accessing a vector database representing a set of inflammation vectors; projecting the set of inflammation vectors and the first inflammation vector into n-dimensional space; identifying a cluster of inflammation vectors, tagged with a first inflammation-related state, proximal the first inflammation vector in n-dimensional space; associating the first inflammation vector with the first inflammation-related state in response to identifying the cluster of inflammation vectors proximal the first inflammation vector; generating a recommendation for diagnosis of the first patient with the first inflammation-related state; and serving the recommendation to a medical operator.

### 1.5 Variation: Artificial Intelligence Model

In another variation, the method S100 includes: receiving a corpus of retinal scans for a population of users, each retinal scan associated with an inflammation-related state; selecting a first set of retinal scans, in the corpus of retinal scans, characterized by a first inflammation-related state; for each retinal scan in the first set of retinal scans, extracting a first set of inflammation biomarkers from the retinal scan and generating an inflammation vector, in a set of inflammation vectors, based on the first set of inflammation biomarkers; and generating a model configured to receive a retinal scan and assign a diagnosis to the retinal scan based on the set of inflammation vectors.

This variation of the method S100 also includes: capturing a first retinal scan for a first patient; extracting a first constellation of inflammation biomarkers from the first retinal scan; generating a first inflammation vector representing the first constellation of inflammation biomarkers; passing the first inflammation vector to the model; receiving a recommendation for diagnosis of the first patient with a first inflammation-related state from the model; and serving the recommendation to a medical operator.

### 1.6 Retinal Scanning System

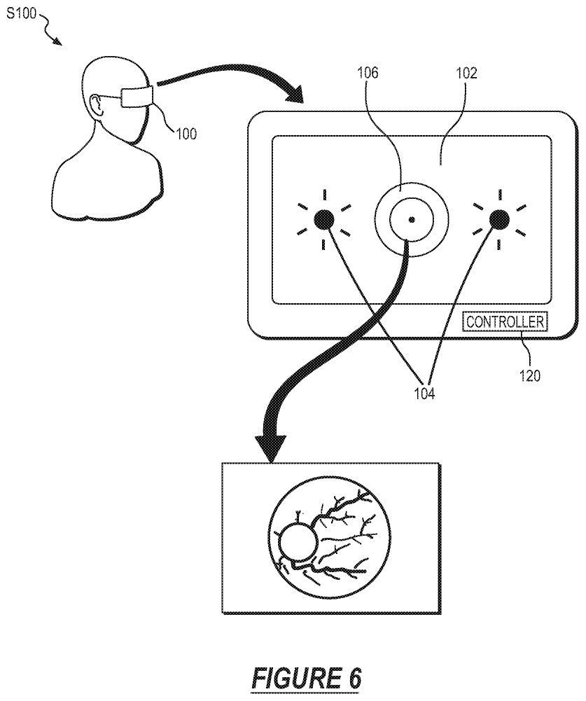

As shown in FIG. 6, a system 100 includes an optical sensor 102 including: an optical emitter 104 configured to illuminate a retina of an eye of a patient; and an optical detector 106 configured to capture a retinal scan of the retina, illuminated by light emitted from the optical emitter 104.

The system 100 also includes an ocular interface 110: including an eye spacer 112; and configured to contact a brow of a patient and maintain a position of the optical sensor 102 relative to the eye of the patient during capture of the retinal scan.

The system 100 further includes a controller 120 configured to: trigger capture of a first retinal scan, for a first patient, by the optical sensor 102; extract a constellation of inflammation biomarkers from the first retinal scan; calculate a first inflammation metric value, for the first inflammation metric, based on a first inflammation function and the constellation of inflammation biomarkers; in response to the first inflammation metric value exceeding a threshold inflammation metric value, generate an inflammation-related state indication corresponding to the first inflammation-related state for the first patient; and transmit the first inflammation metric value and the inflammation-related state indication to an operator portal.

### 2. Applications

Generally, the method S100 can be executed by a computer system, in coordination with a retinal imaging or scanning system, to: capture a retinal scan for a patient; extract constellations of inflammation biomarkers from layers of the retinal scan; calculate inflammation metric values indicative of, or correlating to, inflammation-related states (e.g., metabolic inflammation, cardiovascular inflammation, systemic inflammation, chronic inflammation, acute inflammation, microvascular inflammation, macrovascular inflammation); and generate an inflammation state indication and corresponding recommendations for the patient (e.g., supplement intake, lifestyle changes, seeking medical attention) based on the inflammation metric values.

In particular, the computer system can generate a set of physiologic metrics from retinal images. The computer system can: receive a set of retinal fundus images and/or fluorescein angiography (FA) images captured during a clinical visit; and generate a set of quantitative metrics representing physiologic or pathophysiologic states of a patient. In particular, the computer system can generate a set of metrics including: a neurofunction metric; a metabolic stress metric; a chronic inflammatory load metric; a macrovascular deviation metric; and/or a microvascular deviation metric.

In one variation, the computer system can implement an attention-based neural network architecture configured to internalize spatial decomposition and inter-regional aggregation of retinal features. In particular, in this variation, the computer system can convert the retinal image into a sequence of learned tokens and process the sequence using a transformer-based architecture. In this variation, the computer system can calculate the set of inflammation metrics without explicit tile-level metric calculation and heuristic aggregation across tiles.

In one variation, for a particular clinical visit, the computer system can access: a set of retinal fundus images; a set of fluorescein angiography images captured during the clinical visit; wearable-derived physiologic data captured during the clinical visit; and/or metadata including device type, field-of-view, pixel resolution, and demographic attributes. The computer system can preprocess each retinal image by: identifying a retinal region to exclude non-retinal borders; executing illumination normalization and color standardization; executing scale normalization based on anatomical priors (e.g., optic disc scale estimation); generating an image quality embedding representing resolution, clarity, and stability; and generating a device or domain embedding to compensate for inter-device variability.

The computer system can then convert each retinal image into a sequence of learned tokens (e.g., segments, tiles, regions), such as based on a projection mechanism including convolutional patch embedding, overlapping patch embedding, and/or hierarchical multi-resolution token formation. The computer system can enrich tokens with positional encodings, image quality embeddings, and device or domain embeddings. For fluorescein angiography imaging, the computer system can encode temporal phase information as additional token channels or as a separate token stream.

Then, the computer system can process the token sequence based on a multi-layer transformer including self-attention layers configured to integrate global retinal context and local attention layers configured to preserve high-resolution vascular features. Additionally or alternatively, the computer system can implement cross-attention layers to enable interaction between fundus and fluorescein angiography tokens and/or interaction between retinal tokens and wearable-derived physiologic tokens. Through these transformations, the computer system can learn vascular topology, perfusion dynamics, texture irregularities, vessel caliber distributions, and structural relationships without predefined regional partitions.

For each inflammation metric in a set of inflammation metrics, the computer system can introduce a learned metric query token corresponding to a specific physiologic construct. For example, the computer system can define: a neurofunction query token; a metabolic stress query token; a chronic inflammatory load query token; a macrovascular deviation query token; and a microvascular deviation query token.

In one example, the computer system can define the neurofunction metric representing learned retinal correlates associated with neural integrity, including patterns in microvascular density, macular region structure, vessel branching organization, and/or perfusion characteristics. In another example, the computer system can define the metabolic stress metric representing learned correlates associated with metabolic dysregulation, including vessel caliber variation, tortuosity patterns, perifoveal density alterations, and/or texture changes associated with metabolic stress states. In another example, the computer system can define the chronic inflammatory load metric representing patterns consistent with sustained microvascular irregularity, vascular wall changes, capillary dropout signatures, and/or perfusion heterogeneity. In yet another example, the computer system can define the macrovascular deviation metric representing deviation from normative macrovascular topology including large vessel tortuosity, branching asymmetry, caliber variability, and arteriovenous relationship anomalies. The microvascular deviation metric can represent deviation in capillary-level density, distribution uniformity, and/or small-vessel branching structure inferred from fundus or fluorescein angiography patterns.

The computer system can then normalize each metric to a population reference distribution, expressed as a percentile, and/or accompanied by a predictive uncertainty estimate. For example, the computer system can associate each metric with: a clinician grading schema; laboratory measurements; a validated questionnaire; a longitudinal clinical endpoint; and/or a population reference distribution.

In one variation, the computer system can encode wearable-derived physiologic signals (e.g., heart rate variability, sleep parameters, glucose-related data, activity load) as wearable tokens and integrate these tokens with retinal tokens through cross-attention. Accordingly, the computer system can enable contextual adjustment of metabolic and inflammatory metrics, improve robustness to transient physiologic states, and enhance longitudinal modeling.

Therefore, by internalizing spatial decomposition and aggregation of retinal features within an attention-based architecture and extracting physiologic construct-specific metric query outputs, the computer system can generate descriptive wellness metrics, replace or supplement blood biomarkers, and derive surrogate endpoints from retinal imagery.

### 2.1 Retina Scan Segmentation

In particular, the computer system can: capture or receive a retinal scan; segment the retinal scan into arrays of tiles, per layer of the retinal scan; detect constellations of inflammation biomarkers within each tile in each array of tiles of the retinal scan; calculate intra-tile metrics, inter-tile metrics, and an inter-layer metric representing spatial distribution of the inflammation biomarkers and relationships between these inflammation biomarkers; and calculate a first inflammation metric value based on a first inflammation function and the constellation of inflammation biomarkers. Because distinct inflammation-related states correspond to distinct spatial configurations of inflammation biomarkers, the computer system can calculate multiple inflammation metric values for distinct inflammation-related states from a single retinal scan.

In one implementation, the computer system can generate inflammation functions configured to map constellations of inflammation biomarkers to inflammation metric values based on a corpus of historical retinal scans. In particular, during a training time period, the computer system can access a corpus of historical retinal scans tagged with reference values of inflammation metrics (e.g., one inflammation metric, a set of inflammation metrics); extract constellations of inflammation biomarkers from the historical retinal scans; and generate inflammation functions relating constellations of inflammation biomarkers to corresponding inflammation metrics.

In one implementation, in response to a first inflammation metric value exceeding a threshold inflammation metric value, the computer system can generate an inflammation state indication corresponding to a first inflammation-related state and serve the inflammation metric value and the inflammation state indication to an operator portal. Additionally or alternatively, the computer system can generate recommendations based on the inflammation metric value, including recommendations relating to supplements, dietary changes, lifestyle changes, and/or consultation with a medical professional.

Additionally, the computer system can update inflammation functions over time based on new retinal scans tagged with reference values of inflammation metrics. In particular, the computer system can refine parameters of existing inflammation functions and/or generate new inflammation functions for new inflammation-related states without retraining previously generated inflammation functions. Therefore, the computer system can expand and refine a function library over time while maintaining continuity of previously defined inflammation metrics.

Additionally or alternatively, the computer system can coordinate with a mobile retinal scanning system to enable repeated capture of retinal scans over time. The computer system can then derive differences between inflammation metric values across time periods to detect progression or regression of an inflammation-related state and adapt recommendations for the patient based on temporal changes in the inflammation metric values.

### 2.1.1 Inter-Tile and Intra-Tile Metrics

In one implementation, the computer system can segment a retinal scan into a set of layers and, for each layer, segment the layer into an array of tiles of a defined tile density. For each tile, the computer system can extract a constellation of inflammation biomarkers and calculate an intra-tile metric representing spatial patterns of inflammation biomarkers within the tile. The computer system can further calculate a set of inter-tile metrics representing relationships between intra-tile metrics of tiles in the array of tiles. Accordingly, the computer system can: calculate intra-tile metrics representing localized structural organization of inflammation biomarkers; and calculate inter-tile metrics representing coordination, dispersion, and regional dominance of inflammation biomarkers across the retinal scan.

By segmenting the retinal scan into tiles and calculating intra-tile metrics and inter-tile metrics, the computer system can transform raw detections of inflammation biomarkers into a structured spatial representation of inflammation across the retina. In particular, the computer system can distinguish localized anomalies confined to a subset of tiles from coordinated spatial patterns preserved across multiple tiles and layers. Therefore, the computer system can calculate inflammation metric values based on presence of inflammation biomarkers and spatial organization of these inflammation biomarkers across regions of the retinal scan, thereby enabling detection of systemic inflammation-related states reflected in coordinated retinal patterns.

Therefore, by extracting constellations of inflammation biomarkers from retinal scans, structuring these constellations through layer-specific tiling and intra-tile, inter-tile, and inter-layer metrics, and mapping these structured spatial representations to inflammation metric values via inflammation functions, the computer system can non-invasively quantify and monitor systemic inflammation-related states of a patient from retinal imagery and generate corresponding inflammation state indications and recommendations.

### 2.2 Variation: Vector Embeddings+AI Model

In one variation, the method S100 can be executed by a remote computer system in coordination with a retinal scanning system to: capture a retinal scan for a patient; detect a set of biomarkers indicating inflammation elsewhere in a body (e.g., global and/or localized outside of the retina) of the patient; and generate or predict diagnosis that produces or is correlated with inflammation—elsewhere in the body—indicated by biomarkers in the retinal scan.

In particular, the computer system can: capture or receive a retinal scan depicting a network of blood vessels; and detect many biomarkers (e.g., inflammation biomarkers) within this network of blood vessels (e.g., blood vessel widths, profiles, sizes; capillary locations and sizes; junction sizes and geometries). Additionally, different inflammation-related states may produce distinct inflammation patterns within the body, which result in different combinations of changes—or biomarkers—in a network of blood vessels in the retina, and multiple (e.g., many) inflammation-related states may be represented in different combinations of biomarkers in this network of blood vessels in the retina. Therefore, the computer system generates, stores, maintains, and/or implements a model (e.g., CNN, artificial intelligence, vector database, image templates) configured to distinguish and predict multiple (or many) different inflammation-related states in a population of patients based on retinal scans of their eyes.

For example, the computer system can access a corpus of retinal scans from a patient population and, for each retinal scan in the corpus of retinal scans: detect a network of blood vessel (or other biomarker) features in the retinal scan; represent the network of blood vessels in an embedding (e.g., vector); label this embedding with a diagnosis (or multiple diagnoses); and store these embeddings in a vector (or other searchable) database.

Then, the computer system can: receive a first retinal scan for a (new) patient; detect or extract a set of blood vessel (or other biomarker) features from the first retinal scan; represent the first retinal scan as an embedding based on the set of blood vessel features; implement nearest neighbor or clustering techniques to find an embedding or a group of embeddings nearest the embedding of the first retinal scan; and, thereby, map a diagnosis (or multiple diagnoses) from label(s) in the embedding or the group of embeddings to the (new) patient.

Additionally, the computer system can update this vector database over time as the computer system receives (new) retinal scans tagged with diagnoses. In particular, the computer system can: update and/or correct diagnosis labels for extant embeddings to thereby correct erroneous diagnoses; aggregate additional embeddings with the same diagnosis labels into the vector database to strengthen these diagnosis modules for new patients; and/or aggregate additional embeddings with additional diagnosis labels to generate new diagnosis modules in the vector database without retraining extant diagnoses. Therefore, the same vector database can be updated and expanded over time to thereby refine diagnosis modules and to support a wider count and range of diagnoses.

Therefore, the computer system can execute Blocks of the method S100 to scan a patient for multiple inflammation-related states, normally tested for via invasive and/or expensive testing, in a non-invasive and quick procedure. Additionally, the computer system can execute Blocks of the method S100 to analyze a retinal scan, for a patient, to derive non-localized diagnosis data (e.g., inflammation patterns within the retina may indicate inflammation patterns corresponding to inflammation of particular sites within the body and/or particular inflammation-related state indications).

Additionally or alternatively, the computer system can coordinate with a mobile retinal scanning system (e.g., a handheld device, a headset) to enable a patient—or other medical operator—to quickly capture a retinal scan for the patient. Therefore, the mobile retinal scanning system can enable the patient to capture multiple retinal scans over time to thereby enable the computer system to track progression of the patient-via inflammation signals and/or other biomarkers-over time.

### 2.3 Variation: Retinal Fingerprints

In one variation, the computer system can: receive a corpus of retinal scans of a population of users, each retinal scan tagged with a particular inflammation-related state; identify a set of retinal scans, in the corpus of retinal scans, characterized by a first inflammation-related state; for the set of retinal scans, derive a template retinal fingerprint representing indicators, such as relationships between and/or locations of biomarkers detected in these retinal scans, for the first inflammation-related state; and populate an inflammation-related state module, for the first inflammation-related state, with the template retinal fingerprint.

In one implementation, the computer system can: receive a set of retinal scans for a first patient; extract a first set of features, such as biological markers (hereinafter “biomarkers”) including blood vessel geometry, optic disc geometry and/or location, etc.; derive a retinal fingerprint representing biomarker patterns, locations, and relationships between biomarkers detected in the first set of features; evaluate the retinal fingerprint according to a first inflammation-related state module, such as based on detecting correlations between the retinal fingerprint and a template retinal fingerprint for the first inflammation-related state module; and, in response to a correlation between the retinal fingerprint and the template retinal fingerprint exceeding a threshold correlation, associate the first inflammation-related state with the patient and transmit the retinal fingerprint, annotated with the first inflammation-related state, to an operator (e.g., medical professional, doctor, nurse) to prompt the operator to confirm the first inflammation-related state for the patient.

In one application of the method S100, a retinal scanning system can capture a first retinal scan—such as including a series of scans for each eye of a patient—and transmit the first retinal scan to a remote computer system. The remote computer system can then: extract biomarkers (e.g., 100, 1000) from the first retinal scan to derive a retinal fingerprint, representing locations and relationships between these biomarkers; access a set of inflammation-related state modules including a set of template retinal fingerprints representing diagnostic criteria-such as biomarker indicators for a particular inflammation-related state and/or set of inflammation-related states; calculate a set of metrics (e.g., 6, 10) for the first retinal scan, the set of metrics representing correlations between biomarkers in the set of template retinal fingerprints and the first retinal scan; calculate a correlation between the set of metrics and a target set of metrics for the particular inflammation-related state; and calculate an inflammation-related state prediction score—representing likelihood of the inflammation-related state for the patient and/or severity of the inflammation-related state for the patient—based on the correlation between the set of metrics and a target set of metrics for the particular inflammation-related state.

Accordingly, in the foregoing application, the computer system can execute Blocks of the method S100 in cooperation with a retinal scanning system to capture retinal scans for a patient and derive an holistic interpretation of features detected in these retinal scans rather than simply presence and/or absence of indicators to thereby derive a likelihood of diagnosis for a patient while avoiding invasive diagnostic testing and maintaining accurate, near real-time recommendations for medical professionals and/or patients.

The method S100 is described herein as executed by a remote computer system (e.g., a remote server, hereinafter a “computer system”). However, Blocks of the method S100 can be executed by one or more entities accessing the network, by a local computer system, or by any other computer system—hereinafter a “system.”

### 3. Terms

Generally, a “tile” or a “segment” is referred to herein as a region of a retinal scan, corresponding to a portion of a layer of the retinal scan. In one implementation, a tile can correspond to a region defined by a rectilinear grid (e.g., orthogonal grid) forming a linear array of rectilinear square tiles. However, a tile may define any other geometry, such as triangular tiles (e.g., a projected triangular mesh), hexagonal tiles (e.g., a cellular lattice structure), annular or circular tiles (e.g., concentric ring structures), radial segments, polar grids, irregular meshes, adaptive grids, and/or tiles defining geometries defined according to location within the retinal scan. In one variation, a “tile” or a “segment” may refer to a (learned) token. The computer system can input learned tokens, representing segments of the retinal scan, into an artificial intelligence model for calculation of inflammation metrics based on relationships between biomarkers indicated in these tokens.

Generally, an “inflammation-related state” is referred to herein as a physiological or pathological condition of a patient associated with systemic and/or localized inflammation and represented by inflammation metrics derived from inflammation biomarkers detected in a retinal scan. For example, an inflammation-related state may refer to metabolic inflammation, cardiovascular inflammation, systemic inflammation, chronic inflammation, acute inflammation, microvascular inflammation, and/or macrovascular inflammation. However, an inflammation-related state may refer to any other type of inflammation-relation condition, anatomical region, neurological pathology, and/or indication of disease and/or pathology.

Generally, an “inflammation metric” is referred to herein as a quantitative value indicative of a degree, tier, stage, presence, progression, or regression of a corresponding inflammation-related state. In particular, an inflammation metric value may indicate low, moderate, or severe levels of inflammation of a particular inflammation type.

### 4. System

Generally, the computer system can receive retinal scans captured by a retinal scanning system, including an optical sensor 102, an ocular interface 110, a controller 120, and/or an operator interface.

In particular, a system 100 can include an optical sensor 102 including: an optical emitter 104 configured to illuminate a retina of an eye of a patient; and an optical detector 106 configured to capture a retinal scan of the retina, illuminated by light emitted from the optical emitter 104. In particular, the optical sensor 102 can capture a multi-layer retinal scan including a set of discrete retinal tissue layers. Additionally or alternatively, the optical sensor 102 can capture a series of depth-resolved image slices and assemble the slices into a multi-layer retinal scan.

The system 100 can also include an ocular interface 110: including an eye spacer 112; and configured to contact a brow of a patient and maintain a position of the optical sensor 102 relative to the eye of the patient during capture of the retinal scan. In particular, during operation of the retinal scanning system, the ocular interface 110 can: contact a brow of a patient to stabilize a position of a head of the patient, relative to the optical sensor 102, to reduce translational and rotational movement during capture of the retinal scan; maintain a fixed distance between the optical emitter 104 and the retina of the patient; and align an optical axis of the optical sensor 102 with a visual axis of the eye to maintain the retina within a field of view of the optical detector 106.

In one implementation, the retinal scanning system can further include an eye-tracking module: arranged proximal the optical sensor 102; and configured to detect micro-saccadic eye movements during capture of the retinal scan.

In another implementation, the retinal scanning system can include an axial positioning actuator configured to dynamically adjust focal depth during retinal scan acquisition to maintain consistent alignment of retinal layer boundaries within a coordinate frame.

In one implementation, a retinal scanning system can: illuminate a retina of a user with light (e.g., white light) emitted from the light source; and, while the retina is illuminated, capture an image of the retina depicting a set of inflammation biomarkers representing a functional and/or structural state of the retina. The retinal scanning system can: capture a first image of a first eye (e.g., left eye) of the patient; and capture a second image of a second eye (e.g., right eye) of the patient.

In one implementation, the retinal scanning system is operable in a scanning ophthalmoscope (hereinafter “SLO”) mode. Additionally or alternatively, the retinal scanning system is operable in an optical coherence tomography (hereinafter “OCT”) mode.

In one implementation, the retinal scanner includes a set of sensors configured to execute the OCT mode. In particular the set of sensors can: emit light (e.g., near-infrared) at a first frequency; and capture a series of images—such as high-resolution, cross-sectional images—of the retina. In this implementation, the set of sensors can: illuminate the retina (or retinas) of a patient with light; detect light reflection off a set of tissue layers in the retina; and generate an image (e.g., two-dimensional image, three-dimensional image) of the retina including the set of tissue layers characterized by a particular thickness (e.g., 0.001 mm). In this implementation, the set of sensors can: capture a first series of images depicting a set of tissue layers (e.g., 10) of the retina; and assemble the first series of images into a composite retinal scan (e.g., three-dimensional representation) including the set of tissue layers.

In one variation, the retinal scanning system can define a head-mounted display (e.g., virtual reality headset) worn by the patient. In this example, the retinal scanning system, via the head-mounted display, can: project a video and/or a series of moving representations perceptible by the patient; track eye movements of the patient as the patient views the video; capture a series of images while the eyes of the patient are tracking movements in the video to capture an holistic representation of the retina of the patient; and assemble the series of images into a three-dimensional representation of the retina.

Additionally, the system 100 can include a controller 120 configured to execute Blocks of the method S100 to: trigger capture of a first retinal scan, for a first patient, by the optical sensor 102; extract a constellation of inflammation biomarkers from the first retinal scan; and calculate a first inflammation metric value, for the first inflammation metric, based on a first inflammation function and the constellation of inflammation biomarkers. In response to the first inflammation metric value exceeding a threshold inflammation metric value, the controller 120 can: generate an inflammation-related state indication corresponding to the first inflammation-related state for the first patient; and transmit the first inflammation metric value and the inflammation-related state indication to an operator portal. In particular, the controller 120 can transmit the first inflammation metric value and the inflammation-related state indication to the operator portal for rendering on a user device associated with an operator of the retinal scanning system.

Additionally, the controller 120 can implement methods and techniques as described herein to normalize retinal scans captured by the optical sensor 102. For example, the controller 120 can receive gaze vector data from the eye-tracking module and compensate for translational and rotational displacement between sequential image frames during assembly of the multi-layer retinal representation.

Therefore, the retinal scanning system—including the optical sensor 102, ocular interface 110, and controller 120—can cooperate to capture multi-layer representations of the retina with sufficient geometric stability and spectral fidelity to enable detection and quantification of structural persistence of inflammation biomarkers across retinal layers as further described below.

### 4.1 Image Capture+Normalization

Generally, the retinal scanning system can: capture a set of retinal scans for a patient; and normalize each retinal scan in the set of retinal scans according to a predefined standard.

In one implementation, the retinal scanning system can: illuminate the retina with white light; and capture a first image of the retina in response to receiving a signal from an operator to capture the first image (e.g., in response to the operator selecting a button to capture the first image).

Additionally or alternatively, the retinal scanning system can: illuminate the retina with white light; detect a first landmark (e.g., optic nerve, fovea) of the retina through an optical lens of the retinal scanning system; and, in response to detecting the first landmark, automatically capture a first image of the retina.

In the foregoing implementations, the retinal scanning system can additionally or alternatively capture a series of images of the retina. For example, the retinal scanning system can capture a series of images of the retina as the eyes of the user move across a range of positions to thereby capture a comprehensive scan of the retina of the user.

In one implementation, the computer system can orient a first retinal scan in response to detecting a deviation in the orientation of the retinal scan. In particular, the computer system can: receive a first retinal scan captured by the retinal scanning system; detect a first landmark in the first retinal scan; and, in response to an orientation of the first landmark deviating from a target orientation of the first landmark, rotate the first retinal scan according to the orientation of the first landmark to thereby enable the computer system to detect biomarkers according to biomarker locations, as described further below.

In another implementation, the computer system can: verify a quality of a first retinal scan; and, in response to the quality of the first retinal scan falling below a target quality, prompt an operator to capture a second retinal scan for the patient. For example, the computer system can: scan the first retinal scan for a first landmark; identify a first resolution of the first retinal scan; and, in response to failure to detect the first landmark and/or in response to the first resolution falling below a threshold resolution, prompt an operator to recapture the first retinal scan for the patient.

### 4.2 Image Processing: Layers+Tiles

Generally, the computer system can: access a first retinal scan, captured by a retinal scanning system, for a first patient; detect a series of layers (e.g., tissue layers, imaging layers, imaging modalities) in the first retinal scan; and segment each layer, in the series of layers, into an array of tiles of a particular tile density.

In particular, the computer system can: segment a first layer of the first retinal scan into a first array of tiles of a first tile density; and segment a second layer of the first retinal scan into a second array of tiles of a second tile density less than the first tile density.

In one implementation, the computer system can define the set of layers, each layer representing a distinct spatial scale of the retinal scan. For example, the computer system can define the set of layers, each layer corresponding to a defined subdivision granularity characterized by a predetermined tile density. For each layer, the computer system can segment the retinal scan into an array of tiles corresponding to the predetermined tile density of that layer.

In one implementation, the computer system can define a tile density for each layer. For example, the computer system can: associate a first layer with a first density of tiles; and associate a second layer with a second density of tiles less than the first density of tiles. Additionally or alternatively, the computer system can define a tile count for each layer. For example, the computer system can: define a first count of tiles for a first layer of the retinal scan; and define a second count of tiles less than the first count of tiles for a second layer of the retinal scan.

In one implementation, the computer system can select a particular tile geometry for each layer in the set of layers. In this implementation, the computer system can: segment the first layer of the first retinal scan into the first array of tiles of a first geometry; and segment the second layer of the first retinal scan into the second array of tiles of a second geometry distinct from the first geometry. For example, the computer system can: segment the first layer of the first retinal scan into the first array of tiles of a first geometry including a rectilinear grid (e.g., orthogonal grid); and segment the second layer of the first retinal scan into the second array of tiles of a second geometry (e.g., a hexagonal array, a radial array of concentric rings, a polar grid, an irregular mesh, an adaptive grid). Similarly, the computer system can segment a layer of the first retinal scan into an array of tiles, each tile in the array of tiles defining a unique shape based on a location (e.g., region) of the tile relative to the retinal scan.

Therefore, by detecting multiple layers of the retinal scan and segmenting each layer into arrays of tiles defined by layer-specific tile densities and geometries, the computer system can generate a multi-scale spatial representation of the retina that enables tile-specific, layer-specific, and cross-layer quantification of spatial distributions of inflammation biomarkers as further described below.

### 5. Inflammation Functions

Generally, the computer system can generate an inflammation function (or “module”) for each inflammation-related state in a population of inflammation-related states, the inflammation function relating spatial relationships between constellations of biomarkers—specific to retinal scans—to a particular inflammation metric (or a set of inflammation metrics) representing the inflammation-related state.

In particular, the computer system can: access a corpus of historical retinal scans for a population of patients, each historical retinal scan in the corpus of historical retinal scans tagged with a reference value of an inflammation metric representing an inflammation-related state; extract constellations of biomarkers from each historical retinal scan in the corpus of historical retinal scans; associate these constellations of biomarkers with the first inflammation metric; and derive an inflammation function relating constellations of biomarkers with the first inflammation metric based on constellations of biomarkers extracted from this corpus of historical retinal scans and corresponding reference values of the first inflammation metric associated with these historical retinal scans.

More specifically, the computer system can access a set of historical retinal scans, each historical retinal scan in the set of historical retinal scans tagged with a reference value of a first inflammation metric representing an inflammation-related state and, for each historical retinal scan in the set of historical retinal scans: extract a first constellation of inflammation biomarkers; and associate the first constellation of inflammation biomarkers with the first inflammation metric.

The computer system can then generate a first inflammation function relating inflammation biomarkers with the first inflammation metric based on associations between constellations of inflammation biomarkers and a corresponding reference value of the first inflammation metric.

In one implementation, for each historical retinal scan in the set of historical retinal scans, the computer system can: extract a first constellation of inflammation biomarkers from a particular layer and/or tile of the historical retinal scan; encode the first constellation of inflammation biomarkers as a structured feature representation including spatial, intensity, and distribution attributes of the inflammation biomarkers; and associate the structured feature representation with the reference value of the first inflammation metric corresponding to the historical retinal scan.

Based on these associations, the computer system can generate a first inflammation function configured to map constellations of inflammation biomarkers to corresponding values of the first inflammation metric. In particular, the computer system can: analyze relationships between structured feature representations of inflammation biomarkers and reference values of the first inflammation metric across the set of historical retinal scans; identify statistical or learned correlations between spatial configurations of inflammation biomarkers and the first inflammation metric; and define the first inflammation function to output an inflammation metric value in response to input of a constellation of inflammation biomarkers extracted from a retinal scan.

In one variation, for a first inflammation-related state, the computer system can: generate a first inflammation function for a first inflammation metric and a second inflammation metric; and combine the first inflammation metric and the second inflammation metric to calculate a first composite inflammation metric representing the first inflammation-related state.

For example, during the training time period, the computer system can access a second set of historical retinal scans, each historical retinal scan in the second set of historical retinal scans tagged with a reference value of a second inflammation metric representing the first inflammation-related state and, for each historical retinal scan in the second set of historical retinal scans: extract a second constellation of inflammation biomarkers; and associate the second constellation of inflammation biomarkers with the second inflammation metric. In this example, the computer system can generate the first inflammation function relating: inflammation biomarkers with the first inflammation metric based on associations between constellations of inflammation biomarkers and a corresponding reference value of the first inflammation metric; and inflammation biomarkers with the second inflammation metric based on associations between constellations of inflammation biomarkers and a corresponding reference value of the second inflammation metric.

Accordingly, in the foregoing variation, the computer system can generate a single inflammation function mapping distinct constellations of inflammation biomarkers, detected in tiles of a particular retinal scan, to a set of inflammation metrics for a particular inflammation-related state.

In another variation, for a first inflammation-related state, the computer system can: generate a first inflammation function for a first inflammation metric; a second inflammation function for a second inflammation metric; and compile (e.g., average) the first inflammation metric and the second inflammation metric to calculate a composite inflammation metric representing the first inflammation-related state.

For example, during the training time period, the computer system can access a second set of historical retinal scans, each historical retinal scan in the second set of historical retinal scans tagged with the first inflammation-related state further represented by a second inflammation metric and, for each historical retinal scan in the second set of historical retinal scans: extract a second constellation of inflammation biomarkers; and associate the second constellation of inflammation biomarkers with the second inflammation metric. The computer system can then generate a second inflammation function relating inflammation biomarkers with the second inflammation metric based on associations between constellations of inflammation biomarkers and the second inflammation metric.

In one variation, the computer system can generate a first inflammation function configured to derive a set of inflammation metrics, each inflammation metric corresponding to an inflammation-related state in a set of inflammation-related states.

In this variation, during the training time period, the computer system can access a second set of historical retinal scans, each historical retinal scan in the second set of historical retinal scans tagged with a reference value of a second inflammation metric representing a second inflammation-related state and, for each historical retinal scan in the second set of historical retinal scans: extract a second constellation of inflammation biomarkers; and associate the second constellation of inflammation biomarkers with the second inflammation metric (e.g., a corresponding reference value of the second inflammation metric). In this variation, the computer system can generate the first inflammation function relating: inflammation biomarkers with the first inflammation metric based on associations between constellations of inflammation biomarkers and a corresponding reference value of the first inflammation metric; and inflammation biomarkers with the second inflammation metric based on associations between constellations of inflammation biomarkers and a corresponding reference value of the second inflammation metric.

Accordingly, by generating the first inflammation function relating constellations of inflammation biomarkers to multiple inflammation metrics corresponding to distinct inflammation-related states, the computer system can derive a set of inflammation metrics from a single retinal scan, thereby enabling concurrent evaluation of such distinct inflammation-related states.

In another variation, the computer system can generate a function library for a set of inflammation-related states.

For example, during the training time period, the computer system can: access a first subset of historical retinal scans tagged with reference values of a first inflammation metric corresponding to a first inflammation-related state; generate a first inflammation function relating constellations of inflammation biomarkers with the first inflammation metric; access a second subset of historical retinal scans tagged with reference values of a second inflammation metric corresponding to a second inflammation-related state; and generate a second inflammation function relating constellations of inflammation biomarkers with the second inflammation metric.

In this example, the computer system can store the first inflammation function and the second inflammation function in the function library, each function associated with its corresponding inflammation-related state.

In particular, the computer system can: generate a first inflammation function for a first inflammation metric for a first inflammation-related state; and generate a second inflammation function, distinct from the first inflammation function, for a second inflammation metric for a second inflammation-related state.

For example, during the training time period, the computer system can access a second set of historical retinal scans, each historical retinal scan in the second set of historical retinal scans tagged with a second inflammation-related state represented by a second inflammation metric and, for each historical retinal scan in the second set of historical retinal scans: extract a second constellation of inflammation biomarkers; and associate the second constellation of inflammation biomarkers with the second inflammation metric. The computer system can then generate a second inflammation function relating inflammation biomarkers with the second inflammation metric based on associations between constellations of inflammation biomarkers and the second inflammation metric.

Additionally or alternatively, for the first inflammation-related state and the second inflammation-related state, the computer system can: generate a first inflammation function configured to output: a first inflammation metric for the first inflammation-related state; and a second inflammation metric for the second inflammation-related state.

In one variation, the computer system can generate an inflammation function library based on patient demographics (e.g., sex, ethnicity, race). For example, the computer system can: access the set of historical retinal scans, each historical retinal scan in the set of historical retinal scans tagged with patient demographic characteristics; select a first subset of historical retinal scans, in the set of historical retinal scans, exhibiting a first set of patient demographic characteristics in Block S180; generate the first inflammation function, as described herein, based on the first subset of historical retinal scans; select a second subset of historical retinal scans, in the set of historical retinal scans, exhibiting a second set of patient demographic characteristics; and generate a second inflammation function, as described herein, based on the second subset of historical retinal scans. In the foregoing example, the computer system can: associate the first inflammation function with the first set of patient demographic characteristics in Block S182; and associate the second inflammation function with the second set of patient demographic characteristics.

Therefore, by generating an inflammation function library—including composite functions, state-specific functions, multi-metric functions, and/or demographic-specific functions—based on associations between constellations of inflammation biomarkers and corresponding reference values of inflammation metrics, the computer system can construct a structured function architecture that enables calculation of inflammation metrics for inflammation-related states from a retinal scan.

### 5.1 Variation: Inflammation Modules

In one variation, the computer system can generate an inflammation module for each inflammation-related state in a population of inflammation-related states, the inflammation function relating spatial relationships between constellations of biomarkers—specific to retinal scans—to a particular inflammation vector (or a set of inflammation vectors) representing the inflammation-related state.

In another implementation, the computer system can: access a corpus of retinal scans for a population of users, each retinal scan tagged with an inflammation-related state; for a first set of retinal scans in the corpus of retinal scans, characterized by a first inflammation-related state, extract a first set of inflammation biomarkers from each retinal scan and generate an inflammation vector, in a set of inflammation vectors, based on the first set of inflammation biomarkers and representing the retinal scan; and store the set of inflammation vectors in a vector database representing a corpus of inflammation-related states. Accordingly, the computer system can generate clusters of embeddings (or “inflammation modules”) representing pathologies and/or inflammation-related states.

In one implementation, the computer system can: receive a corpus of retinal scans for a population of patients, each retinal scan tagged with a particular inflammation-related state; for a subset of retinal scans tagged with a first inflammation-related state, average a subset of retinal scans to derive a composite retinal scan; extract a set of biomarkers from the composite retinal scan; derive an inflammation vector based on the set of biomarkers; and store the first inflammation vector, associated with the first inflammation-related state, in a first module representing the first inflammation-related state.

In particular, in the foregoing implementation, the computer system can: detect a constellation of biomarkers in the composite retinal scan; identify (e.g., tag) locations of biomarkers, in the constellation of biomarkers; derive a set of characteristics (e.g., geometry, foci count, volume, area of locations, density ratios) from the constellation of biomarkers, such as based on the locations of biomarkers; and derive the first inflammation vector based on the set of characteristics.

In one variation, the computer system can populate a module representing a first inflammation-related state with a cluster of inflammation vectors indicating the first inflammation-related state. For example, in this implementation, the computer system can: select a first subset of retinal scans, in a set of retinal scans tagged with the first inflammation-related state and defining homologous characteristics and/or analogous biomarkers; and, for each retinal scan in the first subset of retinal scans identify a cluster of biomarkers indicating the first inflammation-related state, identify a set of characteristics for the cluster of biomarkers such as including geometry and/or location (e.g., pixel location) of biomarkers in the cluster of biomarkers, and generate an inflammation vector based on the set of characteristics for the cluster of biomarkers. The computer system can then implement methods and techniques described herein for each subset of retinal scans, in the set of retinal scans tagged with the first inflammation-related state, to: generate a set of inflammation vectors representing indication of the first inflammation-related state; and store these inflammation vectors in a vector database representing inflammation modules for a set of inflammation-related states.

In the foregoing implementation, the computer system can accordingly generate modules including inflammation vectors for indication of inflammation-related states. Additionally, the computer system can generate modules for indication of inflammation-related states including: a particular range of metrics for biomarkers in the inflammation vectors; and/or a confidence threshold for each inflammation vector in the set of inflammation vectors.

The computer system can repeat the methods and techniques described herein for each inflammation-related state, represented by subsets of retinal scans in the corpus of retinal scans, to derive a set of inflammation vectors representing the set of inflammation-related states.

### 5.2 Inflammation-Related State Module/Function Validation+Updates

Generally, the computer system can: validate each inflammation function in the function library over time; and populate the function library with new inflammation functions while maintaining historical inflammation functions.

In particular, the computer system can implement methods and techniques as described herein to generate a new inflammation function for a new inflammation metric without retraining previously generated inflammation functions.

Therefore, the computer system can expand and refine the function library to incorporate new inflammation metrics and new inflammation-related states without retraining or modifying existing inflammation functions to thereby reduce latency of addition of new inflammation functions to the function library and preserve historical inflammation functions in the function library.

In one variation, the computer system can validate the set of inflammation vectors based on clinical study comparison. In particular, the computer system can: receive a set of test retinal scans, such as from a clinical study; identify a corresponding inflammation vector from the set of inflammation vectors for a first test retinal scan in the set of test retinal scans, the corresponding inflammation vector representing a second inflammation-related state; receive a first tag for the first test retinal scan, such as appended by an operator (e.g., clinical study administrator, principle investigator); and, in response to the first tag differing from the second inflammation-related state, repeat methods and techniques described herein with a second corpus of retinal scans to retrain the computer system for the second inflammation-related state. Additionally or alternatively, in response to the first tag corresponding to the second inflammation-related state, the computer system can validate the set of inflammation vectors for the second inflammation-related state.

In one implementation, the computer system can implement methods and techniques described herein to generate a set of inflammation vectors for a particular inflammation-related state, each inflammation vector in the set of inflammation vectors representing a particular severity, or stage, of the particular inflammation-related state.

For example, the computer system can select a first subset of retinal scans, in a set of retinal scans tagged with the first inflammation-related state of a first severity, and for each retinal scan in the first subset of retinal scans: identify a cluster of inflammation biomarkers indicating the first inflammation-related state of the first severity; identify a set of characteristics for the cluster of inflammation biomarkers, such as including geometry and/or location (e.g., pixel location) of inflammation biomarkers in the cluster of inflammation biomarkers; and generate an inflammation vector based on the set of characteristics for the cluster of inflammation biomarkers and representing the first inflammation-related state of the first severity. The computer system can then implement methods and techniques described herein for each severity of the first inflammation-related state represented by a subset of retinal scans, in the set of retinal scans tagged with the first inflammation-related state, to generate a set of inflammation vectors representing indication of the first inflammation-related state of varying levels of severity and/or stage.

Accordingly, in the foregoing implementation, the computer system can generate and/or update modules for additional inflammation-related states without retraining each existing module to thereby reduce computational load for storage and execution of modules in the set of modules.

### 6. Scan Processing+Inflammation Metrics

Generally, for a first retinal scan for a patient, the computer system can: extract a constellation of biomarkers (e.g., blood vessels, retinal anomalies, ocular manifestations, optic disc, optic nerve, fovea) from the first retinal scan; and derive an inflammation metric from the constellation of biomarkers, the inflammation metric indicative of systemic inflammation within the body of the patient, and relationships between these biomarkers.

In one implementation, the computer system can: segment the first retinal scan into a set of layers; segment each layer into an array of tiles; extract spatial attributes of inflammation biomarkers from each tile in the array of tiles; calculate intra-tile metrics representing local spatial configurations of inflammation biomarkers; calculate inter-tile metrics representing relationships between spatial configurations of inflammation biomarkers across tiles; and derive the inflammation metric based on the intra-tile metrics and the inter-tile metrics.

For example, the computer system can: detect vessel tortuosity, localized reflectivity anomalies, and structural irregularities within tiles of a particular layer; quantify spatial dispersion and clustering of these inflammation biomarkers across the retinal scan; and calculate the inflammation metric proportional to a degree of coordinated spatial disruption represented by the constellation of inflammation biomarkers.

In another implementation, the computer system can select a function, in a function library, based on characteristics of the target patient for calculation of the inflammation metric. In particular, the computer system can: access a set of demographic characteristics of the patient; and select an inflammation function from the function library based on the set of demographic characteristics. In one example, the computer system can: access a set of patient demographic characteristics associated with the first patient in Block S184; and select the first inflammation function in response to the set of patient demographic characteristics corresponding to a target set of patient demographic characteristics associated with the first inflammation function in Block S186.

In one variation, the computer system can: extract a set of biomarkers (e.g., blood vessels, retinal anomalies, ocular manifestations, optic disc, optic nerve, fovea) from the first retinal scan; and derive an inflammation vector from the set of biomarkers, the inflammation vector representing a constellation of biomarkers, in the set of biomarkers, indicative of inflammation within the body of the patient, and relationships between these biomarkers.

In particular, the computer system can: receive a first retinal scan (e.g., a series of retinal scans, two-dimensional representation, three-dimensional representation), such as from the retinal scanning system; detect a landmark feature, such as an optic disc, an optic nerve, a fovea, etc.; extract a set of biomarkers from the first retinal scan based on the landmark feature; evaluate pairs of biomarkers, in the set of biomarkers, to identify a constellation of biomarkers in the first retinal scan; and derive an inflammation vector based on the constellation of biomarkers.

In one example, the computer system can derive the inflammation vector based on presence of particular biomarkers. In particular, the computer system can detect presence of: a cotton wool spot (e.g., fluffy white patches); a retinal hemorrhage; cystoid spaces; and/or exudates (e.g., bright yellow clusters, bright white clusters). The computer system can then generate an inflammation vector including these particular biomarkers. Accordingly, the computer system can select particular biomarkers for generation of a representation of the retinal scan to thereby execute a targeted diagnostic scan on these particular biomarkers, as further described below.

Similarly, the computer system can derive the inflammation vector based on locations of particular biomarkers, such as a first biomarker deviating from a target location for the first biomarker type (e.g., detecting vitritis cells in the anterior vitreous).

In another example, the computer system can derive the inflammation vector based on geometry and/or characteristics of biomarkers in the constellation of biomarkers. In particular, the computer system can: detect each blood vessel, in a set of blood vessels, in the retinal scan; and derive a geometry of each blood vessel in the set of blood vessels, the geometry including a shape of the blood vessel (e.g., tortuosity, curling), a diameter of the blood vessel, a location (e.g., x, y location, x, y, z location), and/or a relative pathway traversed by the blood vessel across the retina. Similarly, the computer system can: detect an optic nerve in the retinal scan; and derive a geometry of the optic nerve, the geometry including a diameter of the optic nerve, a location (e.g., x, y location, x, y, z location), and/or a relative pathway traversed by the optic nerve across the retina.

The computer system can implement methods and techniques described herein for each biomarker detected in the constellation of biomarkers, such as including nerve, tissue, and pigmentation detected in the retinal scan, to thereby derive an inflammation vector representative of each biomarker in the constellation of biomarkers.

In one implementation, the computer system can: apply a filter to the retinal scan of the patient to derive a filtered retinal scan; implement methods and techniques described herein to detect biomarkers in the filtered retinal scan; and derive the inflammation vector based on these biomarkers detected in the filtered retinal scan.

For example, the computer system can: apply a first filter of a first color (e.g., blue) to the first retinal scan; apply a second filter of a second color (e.g., green) to the first retinal scan; apply a third filter of a third color (e.g., red) to the first retinal scan; identify regions of interest for each filtered retinal scan; extract a set of biomarkers from each filtered retinal scan according to the regions of interest; and derive an inflammation vector based on each set of biomarkers.

Accordingly, in the foregoing example, the computer system can derive an inflammation vector representing a comprehensive representation of the first retinal scan, such as by identifying target biomarkers from a corpus of biomarkers (e.g., 1000, 10000, 1000000) represented across a set of filtered retinal scans.

### 6.1 Intra-Tile Metrics

As described above, the computer system can: detect a series of layers in the retinal scan; and segment each layer, in the series of layers, into an array of tiles of a particular tile density. The computer system can then, for each tile in the array of tiles: extract a constellation of inflammation biomarkers from the tile; and calculate an inter-tile metric, representing spatial patterns of inflammation biomarkers in the constellation of inflammation biomarkers.

In one implementation, the computer system can segment a first layer of the first retinal scan into a first array of tiles of a first tile density and, for each tile in the first array of tiles: extract a constellation of inflammation biomarkers from the tile; and calculate an intra-tile metric, in a first set of intra-tile metrics, representing spatial patterns of inflammation biomarkers in the constellation of inflammation biomarkers. The computer system can additionally segment a second layer of the first retinal scan into a second array of tiles of a second tile density less than the first tile density and, for each tile in the second array of tiles: extract a constellation of inflammation biomarkers from the tile; and calculate an intra-tile metric, in a second set of intra-tile metrics, representing spatial patterns of inflammation biomarkers in the constellation of inflammation biomarkers.

For example, for a first tile in the array of tiles in a first layer of a first retinal scan, the computer system can: detect a constellation of biomarkers (e.g., blood vessels); and derive a first attribute metric representing relationships and/or spatial patterns between attributes in the constellation of biomarkers. The computer system can then: repeat methods and techniques as described herein for each tile in the array of tiles in the first layer; and repeat methods and techniques as described herein for each layer in the retinal scan.

In one example, for a first tile in the first array of tiles corresponding to the first layer of the first retinal scan, the computer system can: detect a constellation of inflammation biomarkers including a set of blood vessels and localized retinal anomalies; calculate a first intra-tile metric representing vessel density within the tile; calculate a second intra-tile metric representing vessel smoothness based on curvature variation of vessel segments; calculate a third intra-tile metric representing a length-to-width ratio of detected vessel segments; and calculate a fourth intra-tile metric representing texture variation within the tile. The computer system can repeat this process for each tile in the first array of tiles to generate the first set of intra-tile metrics for the first layer. The computer system can similarly repeat this process for each tile in the second array of tiles corresponding to the second layer to generate the second set of intra-tile metrics.

Accordingly, by extracting constellations of inflammation biomarkers from tiles across multiple layers and calculating attribute metrics representing spatial configurations of these inflammation biomarkers within each tile, the computer system can generate a structured, multi-layer representation of localized inflammation patterns for calculation of inflammation metrics reflective of systemic inflammation within the body of the patient.

Therefore, by calculating intra-tile metrics representing spatial organization of inflammation biomarkers within localized retinal regions, the computer system can transform raw biomarker detections into structured spatial representations that enable quantification of localized structural disruption associated with systemic inflammation.

### 6.2 Inter-Tile Metrics

Generally, the computer system can calculate a set of inter-tile metrics, representing relationships between intra-tile metrics of tiles in the array of tiles, based on the set of intra-tile metrics. In particular, the computer system can calculate an inter-tile metric representing spatial coherence, dispersion, and structural coordination of inflammation biomarkers across distinct regions of the retinal scan.

In one implementation, the computer system can: segment a first layer of the first retinal scan into a first array of tiles of a first tile density; calculate a first set of intra-tile metrics, representing spatial patterns of inflammation biomarkers in constellations of inflammation biomarkers, for the first array of tiles; and calculate a first set of inter-tile metrics, representing relationships between intra-tile metrics of tiles in the first array of tiles, based on the first set of intra-tile metrics.

Additionally, in the foregoing implementation the computer system can: segment a second layer of the first retinal scan into a second array of tiles of a second tile density less than the first tile density; calculate a second set of intra-tile metrics, representing spatial patterns of inflammation biomarkers in constellations of inflammation biomarkers, for the second array of tiles; and calculate a second set of inter-tile metrics, representing relationships between intra-tile metrics of tiles in the second array of tiles, based on the second set of intra-tile metrics.

In one implementation, the computer system can: calculate a variance score for each tile based on dispersion between the intra-tile metric of the tile and intra-tile metrics of other tiles in the array of tiles; and assign a scale factor to each tile inversely proportional to the variance score.

Additionally or alternatively, in response to the variance score exceeding a threshold variance score, the computer system can: exclude the particular tile; and/or assign a reduced scale factor to the particular tile during calculation of inter-tile metrics.

For example, for each tile in the first array of tiles, the computer system can: calculate a first variance score representing dispersion between the intra-tile metric of the tile and intra-tile metrics of tiles in the first array of tiles; and assign a first scale factor, in a first set of scale factors, to the tile inversely proportional to the first variance score. In this example, the computer system can calculate the inter-tile metric, in the first set of inter-tile metrics, based on the first set of intra-tile metrics, each intra-tile metric in the first set of intra-tile metrics weighted by a corresponding scale factor in the first set of scale factors. Additionally, in the foregoing example, for each tile in the second array of tiles, the computer system can: calculate a second variance score representing dispersion between the intra-tile metric of the tile and intra-tile metrics of tiles in the second array of tiles; and assign a second scale factor, in a second set of scale factors, to the tile inversely proportional to the second variance score. The computer system can then calculate the inter-tile metric, in the second set of inter-tile metrics, based on the second set of intra-tile metrics, each intra-tile metric in the second set of intra-tile metrics weighted by a corresponding scale factor in the second set of scale factors.

Therefore, by weighting intra-tile metrics based on variance scores and excluding tiles exhibiting variance scores exceeding the threshold variance score, the computer system can reduce influence of localized artifacts on calculation of inter-tile metrics and generate layer-level metrics reflecting coordinated spatial organization of inflammation biomarkers across the retinal scan.

### 6.3 Inter-Layer Metrics

Generally, the computer system can calculate an inter-layer metric representing structural persistence of inflammation-related biomarker spatial patterns across layers of particular tile densities. In particular, the computer system can calculate an inter-layer metric, representing spatial relationships between constellations of inflammation biomarkers in distinct regions of a retinal scan, for each layer in a set of layers of the retinal scan, based on: the first set of intra-tile metrics; the first set of inter-tile metrics; the second set of intra-tile metrics; and the second set of inter-tile metrics.

The computer system can then calculate the first inflammation metric value, for the first inflammation metric, based on the first inflammation function, the first constellation of inflammation biomarkers, and the inter-layer metric.

Additionally or alternatively, the computer system can define the inter-layer metric as a cross-resolution structural coherence parameter representing persistence of clustering topology, gradient orientation, regional dominance hierarchy, and spatial covariance structure of inflammation biomarkers between the first layer and the second layer of the retinal scan.

In one implementation, the computer system can aggregate weighted intra-tile metrics and inter-tile metrics within each layer to calculate an inter-layer inflammation metric (e.g., a layer-level composite metric). The computer system can further evaluate relationships between inter-layer inflammation metrics, for each layer in the set of layers, to: detect spatial configurations of inflammation biomarkers across layers; and identify recurrent inflammation patterns represented at distinct spatial scales.

In another implementation, the computer system can calculate an inter-layer metric by: mapping the first set of intra-tile metrics and the first set of inter-tile metrics onto the second array of tiles; and quantifying divergence between a first structural distribution of biomarkers defined by the first set of intra-tile metrics and the first set of inter-tile metrics; and a second structural distribution defined by the second set of intra-tile metrics and the second set of inter-tile metrics.

Accordingly, the computer system can calculate the first inflammation metric value proportional to preservation of spatial configurations of inflammation biomarkers across layers of the retinal scan represented by the inter-layer metric.

In one implementation, the computer system can detect a systemic inflammation-related state and/or a localized inflammation-related state based on the inter-layer metric representing persistence of spatial patterns of inflammation biomarkers across layers in the retinal scan. In particular, the computer system can: detect constellations of inflammation biomarkers exhibiting consistent spatial configurations across multiple layers and corresponding to a systemic inflammation-related state; and/or detect constellations of inflammation biomarkers present in a single layer (e.g., not preserved across layers) and corresponding to a localized inflammation-related state.

Therefore, by calculating structural persistence of inflammation-related biomarker spatial patterns across layers of particular tile densities, the computer system can: distinguish constellations of inflammation biomarkers exhibiting coordinated spatial organization across layers from constellations of inflammation biomarkers limited to a single layer; and calculate the first inflammation metric value to reflect a systemic inflammation-related state or a localized inflammation-related state based on the inter-layer metric.

### 6.4 Inflammation-Related State Indication

Generally, the computer system can generate an inflammation-related state indication corresponding to the first inflammation-related state for the first patient in response to the first inflammation metric value exceeding a threshold inflammation metric value. In particular, the computer system can generate a systemic state indication corresponding to systemic presence of the first inflammation-related state for the first patient in response to the first inflammation metric value exceeding a threshold inflammation metric value.

In one implementation, the computer system can generate an inflammation-related state indication, corresponding to the first inflammation-related state, for the first patient in response to the first inflammation metric value and a second inflammation metric value exceeding the threshold inflammation metric value.

For example, during the execution time period, the computer system can: extract a third constellation of inflammation biomarkers from the first retinal scan; calculate a second inflammation metric value, for the second inflammation metric, based on the first inflammation function and the third constellation of inflammation biomarkers; and, in response to the first inflammation metric value and the second inflammation metric value exceeding the threshold inflammation metric value, generating the inflammation-related state indication corresponding to the first inflammation-related state for the first patient.

Additionally or alternatively, the computer system can generate an inflammation-related state indication, corresponding to the first inflammation-related state, for the first patient in response to the first inflammation metric value exceeding a first threshold inflammation metric value and a second inflammation metric value exceeding a second threshold inflammation metric value.

For example, during the execution time period, the computer system can: extract a third constellation of inflammation biomarkers from the first retinal scan; calculate a second inflammation metric value, for the second inflammation metric, based on the first inflammation function and the third constellation of inflammation biomarkers; and, in response to the first inflammation metric value exceeding a first threshold inflammation metric value and the second inflammation metric value exceeding a second threshold inflammation metric value, generate the inflammation-related state indication corresponding to the first inflammation-related state for the first patient.

In another implementation, the computer system can derive severity (e.g., tier, level, stage) of the inflammation-related state for the patient based on defined metric bands.

For example, the computer system can: define a first range of inflammation metric values (e.g., one to three), for the first inflammation metric, corresponding to a first inflammation tier (e.g., healthy, not severe, not concerning) of the first inflammation-related state in Block S170; define a second range of inflammation metric values (e.g., four to five), for the first inflammation metric, corresponding to a second inflammation tier (e.g., moderate), exceeding the first inflammation tier, of the first inflammation-related state in Block S172; and define a third range of inflammation metric values, for the first inflammation metric, corresponding to a third inflammation tier (e.g., severe, extreme), exceeding the second inflammation tier, of the first inflammation-related state in Block S174. In this example and in response to the first inflammation metric value falling within the second range of inflammation metric value, the computer system can generate the inflammation-related state indication corresponding to the second inflammation tier of the first inflammation-related state for the first patient.

Additionally or alternatively, the computer system can: access a set of ranges of inflammation metrics corresponding to particular inflammation-related state tiers from a model (e.g., an artificial intelligence model, a natural language model). The computer system can then implement methods and techniques as described herein to assign a particular inflammation-related state tier to a particular patient based on an inflammation metric associated with the particular patient falling within a particular range of inflammation metrics corresponding to the particular inflammation-related state tier.

### 6.5 Retinal Scan Regions+Inflammation Metrics

In one implementation, the computer system can weight particular regions of the retinal scan based on a target inflammation-related state. In particular, the computer system can: access a set of regions associated with the first inflammation-related state; for a first retinal scan, detect a first region in the set of regions; access a first scale factor corresponding to the first region; and assign the first scale factor to inflammation metrics generated for tiles in the first region of the first retinal scan.

In one example, the computer system can access a set of regions associated with the first inflammation-related state in Block S152. In this example and for the first array of tiles, the computer system can: detect a first subarray of tiles in a first region, corresponding to a region in the set of regions, in the first layer of the first retinal scan in Block S154; assign a first scale factor to the first subarray of tiles in Block S156; detect a second subarray of tiles in a second region, excluded from the set of regions, in the first layer of the first retinal scan in Block S158; and assign a second scale factor, falling below the first scale factor, to the second subarray of tiles in Block S159.

In this example, for the second array of tiles, the computer system can: detect a third subarray of tiles in the first region in the second layer of the first retinal scan; assign the first scale factor to the third subarray of tiles; detect a fourth subarray of tiles in the second region, excluded from the set of regions, in the second layer of the first retinal scan; and assign the second scale factor, falling below the third scale factor, to the fourth subarray of tiles.

The computer system can repeat methods and techniques as described herein for: each subarray of tiles representing regions in the set of regions; and each layer in the set of layers of the retinal scan.

Accordingly, in the foregoing example, the computer system can weight particular regions of the retinal scan that may exhibit higher correspondence to the target inflammation-related state relative to regions of the retinal scan exhibiting lower correspondence to the target inflammation-related state.

Therefore, the computer system can localize intra-tile, inter-tile, and inter-layer metric calculation to: weight regions-of-interest relative to a target inflammation-related state for each retinal scan; and preserve computational resources for calculation of relevant inflammation metrics.

### 6.6 Multi-State Analysis

Generally, the computer system can repeat methods and techniques as described herein for each state in a set of inflammation-related states.

For example, during the execution time period, the computer system can: extract a third constellation of inflammation biomarkers from the first retinal scan; calculate a second inflammation metric value, for the second inflammation metric, based on the first inflammation function and the third constellation of inflammation biomarkers; and, in response to the second inflammation metric value exceeding the threshold inflammation metric value, generate a second inflammation-related state indication corresponding to the second inflammation-related state for the first patient and serve the second inflammation metric value and the second inflammation-related state indication to the operator portal.

The computer system can then repeat this method for each state in a set of inflammation-related states (e.g., seven target inflammation-related states, inflammation-related states designated by a retinal scanning system operator).

In one implementation, the computer system can access a first inflammation function configured to output: a first inflammation metric for a first inflammation-related state; and a second inflammation metric for a second inflammation-related state. Additionally or alternatively, the computer system can: access a first inflammation function configured to output a first inflammation metric for a first inflammation-related state; and access a second inflammation function configured to output a second inflammation metric for a second inflammation-related state.

Additionally or alternatively, the computer system can: access a first inflammation function configured to output a first inflammation metric and a second inflammation metric for a first inflammation-related state; and access a second inflammation function configured to output a third inflammation metric and a fourth inflammation metric for a second inflammation-related state.

In one variation, the computer system can implement a first threshold inflammation metric value for each inflammation-related state in the set of inflammation-related states. In particular, the computer system can: extract a third constellation of inflammation biomarkers from the first retinal scan; calculate a second inflammation metric value, for the second inflammation metric, based on the first inflammation function and the third constellation of inflammation biomarkers; and, in response to the second inflammation metric value exceeding the threshold inflammation metric value, generate a second inflammation-related state indication corresponding to the second inflammation-related state for the first patient and serve the second inflammation metric value and the second inflammation-related state indication to the operator portal.

Additionally or alternatively, the computer system can access a set of threshold inflammation metric values, each threshold inflammation metric value corresponding to a particular inflammation-related state in the set of inflammation-related states. In particular, the computer system can: access a second threshold inflammation metric value corresponding to the second inflammation-related state; and, in response to the second inflammation metric value exceeding the threshold inflammation metric value, generate the second inflammation-related state indication corresponding to the second inflammation-related state for the first patient.

Therefore, in the foregoing implementations, the computer system can execute concurrent derivation of inflammation metrics for each inflammation-related state in a set of target inflammation-related states to simultaneously detect associations between the patient and inflammation-related states-such as severity levels of each inflammation-related state exhibited by the patient.

### 6.7 Variation: Retinal Fingerprints

Additionally or alternatively, the computer system can implement the methods and techniques described herein to derive a set of template retinal fingerprints for each inflammation-related state module. For example, for a first retinal scan, the computer system can: extract a set of biomarkers (e.g., blood vessels, retinal anomalies, ocular manifestations, optic disc, optic nerve, fovea) from the first retinal scan; and derive a retinal fingerprint from the set of biomarkers, the retinal fingerprint representing a constellation of biomarkers, in the set of biomarkers, indicative of inflammation-related states, and relationships between these biomarkers.

### 6.8 Variation: Inflammation Vector Module Execution

In one implementation, the computer system can: execute a module, in the set of modules and including an inflammation vector and representing an inflammation-related state and, in particular, inflammation indicators of the inflammation-related state, for the inflammation vector for the patient to derive the set of metrics and/or calculate a correlation between the inflammation vectors and the inflammation vector for the patient; and, in response to the correlation exceeding a threshold correlation, recommend diagnosis of the inflammation-related state for the patient.

In this implementation, the computer system can calculate a set of metrics based on correlation between biomarkers in the first inflammation vector and the inflammation vector. In particular, the computer system can: select a first region of the first inflammation vector according to the inflammation-related state module; calculate a first metric, in a set of metrics, based on correlation between the first region of the first inflammation vector and the first corresponding region of the first inflammation vector; select a first target biomarker for the first inflammation vector according to the inflammation-related state module; calculate a second metric, in the set of metrics, based on correlation between the first target biomarker of the first inflammation vector and the first corresponding target biomarker of the first inflammation vector; select a second region of the first inflammation vector according to the inflammation-related state module; calculate a third metric, in a set of metrics, based on correlation between the second region of the first inflammation vector and the second corresponding region of the first inflammation vector; select a second target biomarker for the first inflammation vector according to the inflammation-related state module; and calculate a fourth metric, in the set of metrics, based on correlation between the second target biomarker of the first inflammation vector and the second corresponding target biomarker of the first inflammation vector.

Accordingly, based on the inflammation-related state module and the inflammation vector, the computer system can selectively calculate metrics for the first inflammation vector.

In the foregoing implementation, the computer system can calculate a diagnostic score based on the diagnostic and the set of metrics, the diagnostic score representing a likelihood of expression of the inflammation-related state represented by the inflammation-related state module.

In one example, the computer system can average the set of metrics to calculate the diagnostic score. In a similar example, the computer system can: access a set of weights, each weight associated with each metric according to the inflammation-related state module; and calculate the diagnostic score based on the set of metrics and the set of weights.

In one variation, the computer system can project the set of inflammation vectors—representing a set of inflammation-related states—and the inflammation vector into n-dimensional space, and identify a nearest-neighbor inflammation vector, or a cluster of inflammation vectors, to the inflammation vector. In response to identifying the nearest-neighbor to the inflammation vector, the computer system can: extract an inflammation-related state associated with the nearest-neighbor inflammation vector (or cluster of inflammation vectors), and recommend a diagnosis of the inflammation-related state to a medical operator.

In one implementation, the computer system can select an inflammation-related state module based on an operator input indicating a particular inflammation-related state associated with the inflammation-related state module, such as the operator input indicating the retinal scan as a test for the particular inflammation-related state.

In yet another variation, the computer system can execute a model including the set of inflammation-related state modules on an inflammation vector and/or a set of inflammation vectors. In this variation, in response to absence of selection of a particular inflammation-related state, the computer system can execute each inflammation-related state module in a set of inflammation-related state modules for the particular inflammation vector. In particular, the computer system can implement methods and techniques described herein to: execute each inflammation-related state module, in the set of inflammation-related state modules, for the first inflammation vector; derive a set of diagnostic scores for the first inflammation vector based on the set of inflammation-related state modules; select a first diagnostic score from the set of diagnostic scores characterized by a highest score in the set of diagnostic scores, the highest score representing a highest likelihood of inflammation-related state characterized by the first inflammation-related state module associated with the first diagnostic score; and recommend diagnosis of the first inflammation-related state associated with the first inflammation-related state module.

In one example, the computer system can access a first inflammation-related state module representing risk of pre-term birth, the first inflammation-related state module including a first template inflammation vector including a set of target biomarkers, such as including arteriolar narrowing, retinal hemorrhages, cotton wool spots, and/or vascular tortuosity.

In another example, the computer system can access a second inflammation-related state module representing risk of endometriosis, the second inflammation-related state module including a second inflammation vector including a second set of target biomarkers, such as including microvascular alterations (e.g., capillary density changes, tortuosity), choroidal or retinal thickness changes (e.g., due to hormonal fluctuations, due to cytokine-driven inflammation), and/or neuroinflammation indicators (e.g., RNFL thinning, microglial activation in systemic autoimmune inflammation-related states).

Accordingly, in the foregoing implementations, the computer system can calculate a correlation and/or set of metrics for a particular inflammation vector, associated with a patient, to thereby identify an inflammation-related state, or a likelihood of inflammation-related state, for the patient, to accordingly recommend diagnosis of the inflammation-related state.

Therefore, the computer system can identify biomarkers that may be otherwise indiscernible and/or obfuscated from a retinal scan to thereby calculate the diagnosis score to enable the medical professional to properly identify the inflammation-related state and enact a treatment plan, based on the inflammation-related state, for the patient.

### 6.9 Confidence Scores

In the foregoing implementations, the computer system can additionally calculate a confidence score for the inflammation-related state indication. In particular, the computer system can calculate a confidence score, representing confidence in the inflammation-related state indication inversely proportional to variance scores associated with tiles in the first array of tiles and the second array of tiles in Block S150; and generate the inflammation-related state indication corresponding to the first inflammation-related state for the first patient in response to the confidence score exceeding a threshold confidence score.

For example, the computer system can: aggregate a set of variance scores-representing dispersion between the intra-tile metric of the tile and intra-tile metrics of tiles in the first array of tiles—into a composite variance score; and calculate the confidence score inversely proportional to the composite variance score. Additionally or alternatively, the computer system can: aggregate a set of variance scores—representing dispersion between constellations of inflammation biomarkers across layers of the retinal scan—into a composite variance score; and calculate the confidence score inversely proportional to the composite variance score.

In one variation, the computer system can calculate the confidence score proportional to retinal scan resolution. For example, the computer system can: access a first resolution of the first retinal scan; and calculate the confidence score proportional to the first resolution of the first retinal scan.

Additionally or alternatively, the computer system can calculate a confidence score for each layer in the set of layers and proportional to a tile density of the layer. For example, the computer system can: access a first tile density of a first layer of the first retinal scan; calculate a first confidence score proportional to the first tile density; access a second tile density, falling below the first tile density, of a second layer of the first retinal scan; and calculate a second confidence score proportional to the second tile density.

In one variation, the computer system can calculate a first confidence score for a first inflammation vector and a first set of inflammation vectors according to a count of vectors proximal the first inflammation vector in n-dimensional space. Additionally or alternatively, the computer system can calculate a first confidence score for the first inflammation vector and a first set of inflammation vectors—tagged with a particular inflammation-related state—according to a count of vectors in the first set of inflammation vectors.

Additionally or alternatively, the computer system can: project the first inflammation vector and the corpus of inflammation vectors—representing a set of inflammation-related states—into n-dimensional space; define a boundary for each cluster of inflammation vectors, in the corpus of inflammation vectors, tagged with a particular inflammation-related state; and, in response to detecting intersection between the first inflammation vector and a first boundary associated with a first cluster of inflammation vectors for a first inflammation-related state, calculate a first confidence score based on a volume of intersection between the first inflammation vector and the first boundary.

In one variation, the computer system can repeat methods and techniques as described herein in response to the confidence score falling below the threshold confidence score. Additionally or alternatively, the computer system can serve the inflammation metrics and corresponding confidence scores to the operator portal to enable the operator to confirm or reject inflammation metric calculation responsive to corresponding confidence scores. In this variation, the computer system can selectively repeat methods and techniques as described herein responsive to operator feedback.

### 7. Variation: Dual-Eye Analysis

In one implementation, the computer system can: access a first retinal scan of a first eye (e.g., a left eye) of a patient; access a second retinal scan of a second eye (e.g., a right eye) of the patient; implement methods and techniques as described herein for the first retinal scan and the second retinal scan to derive an inflammation metric value, representing the inflammation-related state, for each retinal scan; combine these inflammation metric values into a composite inflammation metric; and generate the inflammation-related state indication corresponding to the first inflammation-related state for the first patient in response to the composite inflammation metric exceeding the threshold inflammation metric value.

In one implementation, the computer system can calculate a confidence score for the inflammation-related state indication proportional to a count of retinal scans accessible for a particular patient. For example, the computer system can: access a first retinal scan of a first eye (e.g., a left eye) of a patient; implement methods and techniques as described herein to derive a first inflammation metric value for the first retinal scan; in response to the first inflammation metric value exceeding a threshold inflammation metric value, generate an inflammation-related state indication corresponding to the first inflammation-related state for the first patient; and implement methods and techniques as described herein to calculate a first confidence score for the inflammation-related state indication. In this example, the computer system can: access a second retinal scan of a second eye (e.g., a right eye) of the patient; implement methods and techniques as described herein to derive a second inflammation metric value for the second retinal scan; calculate a composite inflammation metric value based on the first inflammation metric value and the second inflammation metric value; in response to the composite inflammation metric value exceeding the threshold inflammation metric value, generate the inflammation-related state indication corresponding to the first inflammation-related state for the first patient; and implement methods and techniques as described herein to calculate a second confidence score for the inflammation-related state indication, the second confidence score exceeding the first confidence score.

In one variation, the computer system can resolve asymmetry between the first retinal scan and the second retinal scan based on bilateral similarity between the first retinal scan and the second retinal scan.

For example, the computer system can: access the first retinal scan of a first eye of the first patient; access a second retinal scan, captured by the retinal scanning system, of a second eye of the first patient; extract a third constellation of inflammation biomarkers from the second retinal scan; calculate a second inflammation metric value, for the first inflammation metric, based on the first inflammation function and the third constellation of inflammation biomarkers; calculate a bilateral similarity metric representing similarity between the first inflammation metric value and the second inflammation metric value in Block S190; and, in response to the bilateral similarity metric exceeding a threshold bilateral similarity metric and in response to the second inflammation metric value exceeding the threshold inflammation metric value, generate the inflammation-related state indication corresponding to the first inflammation-related state for the first patient.

In one variation, the computer system can select a particular inflammation function, from the function library, based on an eye of the retinal scan. For example, the computer system can: detect a first retinal scan is of a left eye of the patient; select a left-eye specific inflammation function from the function library; and implement methods and techniques as described herein based on the left-eye specific inflammation function. Additionally or alternatively, the computer system can: detect that a first retinal scan is of a right eye of the patient; select a right-eye specific inflammation function from the function library; and implement methods and techniques as described herein based on the right-eye specific inflammation function.

In yet another variation, the computer system can: access a first retinal scan of a first eye (e.g., a left eye) of a patient; access a second retinal scan of a second eye (e.g., a right eye) of the patient; average the first retinal scan and the second retinal scan to generate a composite retinal scan; and implement methods and techniques as described herein for the composite retinal scan to derive a composite inflammation metric value, representing the inflammation-related state; and generate the inflammation-related state indication corresponding to the first inflammation-related state for the first patient in response to the composite inflammation metric exceeding the threshold inflammation metric value.

### 8. Variation: Template Retinal Fingerprints

In one variation, the computer system can calculate a set of metrics based on correlation between biomarkers in the first retinal fingerprint and the template retinal fingerprint. In particular, the computer system can: select a first region of the first retinal fingerprint according to the inflammation-related state module; calculate a first metric, in a set of metrics, based on correlation between the first region of the first retinal fingerprint and the first corresponding region of the first template retinal fingerprint; select a first target biomarker for the first retinal fingerprint according to the inflammation-related state module; calculate a second metric, in the set of metrics, based on correlation between the first target biomarker of the first retinal fingerprint and the first corresponding target biomarker of the first template retinal fingerprint; select a second region of the first retinal fingerprint according to the inflammation-related state module; calculate a third metric, in a set of metrics, based on correlation between the second region of the first retinal fingerprint and the second corresponding region of the first template retinal fingerprint; select a second target biomarker for the first retinal fingerprint according to the inflammation-related state module; and calculate a fourth metric, in the set of metrics, based on correlation between the second target biomarker of the first retinal fingerprint and the second corresponding target biomarker of the first template retinal fingerprint.

Accordingly, based on the inflammation-related state module and the template retinal fingerprint, the computer system can selectively calculate metrics for the first retinal fingerprint.

### 9. Inflammation Progression+Feedback Survey

Generally, the computer system can: derive differences between inflammation metrics of a first patient across a series of retinal scans; and predict progression or regression of the first inflammation-related state according to these differences.

In particular, the computer system can: access a first inflammation metric, generated for the patient during a first time period and for a first retinal scan; access a second inflammation metric, generated for the patient during a second time period, succeeding the first time period, and for a second retinal scan; derive a difference between the first inflammation metric and the second inflammation metric; and, in response to the difference exceeding a threshold difference, predict progression of the first inflammation-related state.

In one implementation, during a third time period succeeding the execution time period, the computer system can: access a second retinal scan, captured by the retinal scanning system during the third time period, for the first patient; extract a third constellation of inflammation biomarkers from the second retinal scan; calculate a second inflammation metric value, for the first inflammation metric, based on the first inflammation function and the third constellation of inflammation biomarkers; and derive a difference between the first inflammation metric value and the second inflammation metric value in Block S160.

In response to the difference (e.g., +3) exceeding a threshold difference (e.g., zero), the computer system can: predict progression of the first inflammation-related state for the first patient in Block S162; and serve the second inflammation metric value and progression of the first inflammation-related state of the first patient to the operator portal.

Additionally or alternatively, in response to the difference (e.g., −2) falling below the threshold difference (e.g., zero), the computer system can: predict regression of the first inflammation-related state for the first patient in Block S162; and serve the second inflammation metric value and regression of the first inflammation-related state of the first patient to the operator portal.

In one variation, the computer system can generate a survey, based on the difference between the first inflammation metric and the second inflammation metric, representing behavioral and/or lifestyle changes of the patient. For example, in response to the difference between the first inflammation metric value and the second inflammation metric value exceeding the threshold difference, the computer system can: access a corpus of questions associated with the first inflammation-related state in Block S164; select a set of questions, in the corpus of questions, based on the difference between the first inflammation metric value and the second inflammation metric value in Block S166; generate a survey consisting of the set of questions in Block S167; and serve the survey and a prompt for the first patient to complete the survey to the operator portal in Block S168.

For example, the computer system can select questions relating to recent behavioral changes, dietary changes, supplement usage, and/or other lifestyle factors associated with the first inflammation-related state. In response to patient responses to the survey, the computer system can correlate reported changes with the derived difference between inflammation metric values and refine interpretation of progression or regression of the first inflammation-related state. Additionally or alternatively, the computer system can generate recommendations including supplements, dietary adjustments, lifestyle modifications, and/or consultation with a medical professional based on the difference and the patient responses.

Therefore, by dynamically generating a survey based on differences between inflammation metric values across time periods, the computer system can associate temporal changes in the first inflammation-related state with behavioral changes indicated by the patient and adapt recommended intervention for the first patient accordingly.

### 10. Iterative Feedback

In one implementation, the computer system can: recommend diagnosis of a first inflammation-related state based on the diagnosis score calculated for a first inflammation vector associated with a first patient; prompt an operator (e.g., medical professional, doctor, nurse) to confirm diagnosis of the first inflammation-related state; in response to receiving rejection of the first inflammation-related state from the operator, prompt the operator to input a diagnosed inflammation-related state for the first patient; tag the first inflammation vector with the diagnosed inflammation-related state; and update an inflammation-related state module, associated with the first inflammation-related state, according to the first inflammation vector.

In one variation, the computer system can: in response to receiving rejection of the first inflammation-related state from the operator, prompt the operator to input a diagnosed inflammation-related state for the first patient; tag the first inflammation vector with the diagnosed inflammation-related state; access a second inflammation-related state module associated with the diagnosed inflammation-related state; access a second set of inflammation vectors for the second inflammation-related state module; identify a deviation between the second set of inflammation vectors and a first inflammation vector associated with the first inflammation-related state; and update the inflammation-related state module, associated with the first inflammation-related state, according to the deviation between the second inflammation vector and a first inflammation vector.

Accordingly, in the foregoing implementation, the computer system can: identify deviations between predicted diagnoses and confirmed diagnoses; map these deviations to correlations between the first inflammation vector and the template inflammation vector; and update inflammation-related state modules according to these deviations and correlations.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a patient computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
during a training time period:
accessing a set of historical retinal scans, each historical retinal scan in the set of historical retinal scans tagged with a reference value of a first inflammation metric indicative of a first inflammation-related state;
for each historical retinal scan in the set of historical retinal scans:
extracting a first constellation of inflammation biomarkers; and
associating the first constellation of inflammation biomarkers with the first inflammation metric; and
generating a first inflammation function relating inflammation biomarkers with the first inflammation metric based on associations between constellations of inflammation biomarkers and a corresponding reference value of the first inflammation metric; and
during an execution time period:
accessing a first retinal scan, captured by a retinal scanning system, for a first patient;
extracting a second constellation of inflammation biomarkers from the first retinal scan;
calculating a first inflammation metric value, for the first inflammation metric, based on the first inflammation function and the second constellation of inflammation biomarkers; and
in response to the first inflammation metric value exceeding a threshold inflammation metric value:
generating an inflammation-related state indication corresponding to the first inflammation-related state for the first patient; and
serving the first inflammation metric value and the inflammation-related state indication to an operator portal.

2. The method of claim 1:
further comprising:
segmenting a first layer of the first retinal scan into a first array of tiles of a first tile density;
for each tile in the first array of tiles:
extracting a constellation of inflammation biomarkers from the tile; and
calculating an intra-tile metric, in a first set of intra-tile metrics, representing spatial patterns of inflammation biomarkers in the constellation of inflammation biomarkers; and
calculating a first set of inter-tile metrics, representing relationships between intra-tile metrics of tiles in the first array of tiles, based on the first set of intra-tile metrics;
segmenting a second layer of the first retinal scan into a second
array of tiles of a second tile density less than the first tile density;
for each tile in the second array of tiles:
extracting a constellation of inflammation biomarkers from the tile; and
calculating an intra-tile metric, in a second set of intra-tile metrics, representing spatial patterns of inflammation biomarkers in the constellation of inflammation biomarkers; and
calculating a second set of inter-tile metrics, representing relationships between intra-tile metrics of tiles in the second array of tiles, based on the second set of intra-tile metrics; and
calculating an inter-layer metric, representing systemic persistence of inflammation-related biomarker spatial patterns, based on:
the first set of intra-tile metrics;
the first set of inter-tile metrics;
the second set of intra-tile metrics; and
the second set of inter-tile metrics; and
wherein calculating the first inflammation metric value, for the first inflammation metric, based on the first inflammation function and the second constellation of inflammation biomarkers comprises calculating the first inflammation metric value further based on the inter-layer metric.

3. The method of claim 2:
wherein calculating the inter-tile metric, in the first set of inter-tile metrics, comprises:
for each tile in the first array of tiles:
calculating a first variance score representing dispersion between the intra-tile metric of the tile and intra-tile metrics of tiles in the first array of tiles; and
assigning a first scale factor, in a first set of scale factors, to the tile inversely proportional to the first variance score; and
calculating the inter-tile metric, in the first set of inter-tile metrics, based on the first set of intra-tile metrics, each intra-tile metric in the first set of intra-tile metrics weighted by a corresponding scale factor in the first set of scale factors; and
wherein calculating the inter-tile metric, in the second set of inter-tile metrics, comprises:
for each tile in the second array of tiles:
calculating a second variance score representing dispersion between the intra-tile metric of the tile and intra-tile metrics of tiles in the second array of tiles; and
assigning a second scale factor, in a second set of scale factors, to the tile inversely proportional to the second variance score; and
calculating the inter-tile metric, in the second set of inter-tile metrics, based on the second set of intra-tile metrics, each intra-tile metric in the second set of intra-tile metrics weighted by a corresponding scale factor in the second set of scale factors.

4. The method of claim 3:
further comprising calculating a confidence score, representing confidence in the inflammation-related state indication inversely proportional to variance scores associated with tiles in the first array of tiles and the second array of tiles; and
wherein generating the inflammation-related state indication corresponding to the first inflammation-related state for the first patient comprises:
generating the inflammation-related state indication corresponding to the first inflammation-related state for the first patient in response to the confidence score exceeding a threshold confidence score.

5. The method of claim 2:
further comprising:
accessing a set of regions associated with the first inflammation-related state;
for the first array of tiles:
detecting a first subarray of tiles in a first region, corresponding to a region in the set of regions, in the first layer of the first retinal scan;
assigning a first scale factor to the first subarray of tiles;
detecting a second subarray of tiles in a second region, excluded from the set of regions, in the first layer of the first retinal scan; and
assigning a second scale factor, falling below the first scale factor, to the second subarray of tiles; and
for the second array of tiles:
detecting a third subarray of tiles in the first region in the second layer of the first retinal scan;
assigning the first scale factor to the third subarray of tiles;
detecting a fourth subarray of tiles in the second region, excluded from the set of regions, in the second layer of the first retinal scan; and
assigning the second scale factor, falling below the third scale factor, to the fourth subarray of tiles; and
wherein generating the inflammation-related state indication corresponding to the first inflammation-related state for the first patient comprises generating a systemic state indication corresponding to the first inflammation-related state for the first patient.

6. The method of claim 2:
wherein segmenting the first layer of the first retinal scan into the first array of tiles of the first tile density comprises segmenting the first layer of the first retinal scan into the first array of tiles of a first geometry; and
wherein segmenting the second layer of the first retinal scan into the second array of tiles of the second tile density comprises segmenting the second layer of the first retinal scan into the second array of tiles of a second geometry distinct from the first geometry.

7. The method of claim 1:
further comprising, during the training time period:
accessing a second set of historical retinal scans, each historical retinal scan in the second set of historical retinal tagged with a reference value of a second inflammation metric representing the first inflammation-related state; and
for each historical retinal scan in the second set of historical retinal scans:
extracting a second constellation of inflammation biomarkers; and
associating the second constellation of inflammation biomarkers with the second inflammation metric;
wherein generating the first inflammation function comprises:
generating the first inflammation function relating:
inflammation biomarkers with the first inflammation metric based on associations between constellations of inflammation biomarkers and a corresponding reference value of the first inflammation metric; and
inflammation biomarkers with the second inflammation metric based on associations between constellations of inflammation biomarkers and a corresponding reference value of the second inflammation metric;
further comprising, during the second time period:
extracting a third constellation of inflammation biomarkers from the first retinal scan; and
calculating a second inflammation metric value, for the second inflammation metric, based on the first inflammation function and the third constellation of inflammation biomarkers; and
wherein generating the inflammation-related state indication corresponding to the first inflammation-related state for the first patient comprises:
in response to the first inflammation metric value and the second inflammation metric value exceeding the threshold inflammation metric value, generating the inflammation-related state indication corresponding to the first inflammation-related state for the first patient.

8. The method of claim 1:
further comprising, during the training time period:
accessing a second set of historical retinal scans, each historical retinal scan in the second set of historical retinal scans tagged with a reference value of a second inflammation metric representing a second inflammation-related state; and
for each historical retinal scan in the second set of historical retinal scans:
extracting a second constellation of inflammation biomarkers; and
associating the second constellation of inflammation biomarkers with the second inflammation metric;
wherein generating the first inflammation function comprises:
generating the first inflammation function relating:
inflammation biomarkers with the first inflammation metric based on associations between constellations of inflammation biomarkers and a corresponding reference value of the first inflammation metric; and
inflammation biomarkers with the second inflammation metric based on associations between constellations of inflammation biomarkers and a corresponding reference value of the second inflammation metric; and
further comprising, during the second time period:
extracting a third constellation of inflammation biomarkers from the first retinal scan;
calculating a second inflammation metric value, for the second inflammation metric, based on the first inflammation function and the third constellation of inflammation biomarkers; and
in response to the second inflammation metric value exceeding the threshold inflammation metric value:
generating a second inflammation-related state indication corresponding to the second inflammation-related state for the first patient; and
serving the second inflammation metric value and the second inflammation-related state indication to the operator portal.

9. The method of claim 1, further comprising:
during a third time period succeeding the execution time period:
accessing a second retinal scan, captured by the retinal scanning system during the third time period, for the first patient;
extracting a third constellation of inflammation biomarkers from the second retinal scan;
calculating a second inflammation metric value, for the first inflammation metric, based on the first inflammation function and the third constellation of inflammation biomarkers;
deriving a difference between the first inflammation metric value and the second inflammation metric value; and
in response to the difference exceeding a threshold difference:
predicting progression of the first inflammation-related state for the first patient; and
serving the second inflammation metric value and progression of the first inflammation-related state of the first patient to the operator portal.

10. The method of claim 9, further comprising:
in response to the difference between the first inflammation metric value and the second inflammation metric value exceeding the threshold difference:
accessing a corpus of questions associated with the first inflammation-related state;
selecting a set of questions, in the corpus of questions, based on the difference between the first inflammation metric value and the second inflammation metric value;
generating a survey consisting of the set of questions; and
serving the survey and a prompt for the first patient to complete the survey to the operator portal.

11. The method of claim 1:
further comprising:
defining a first range of inflammation metric values, for the first inflammation metric, corresponding to a first inflammation tier of the first inflammation-related state;
defining a second range of inflammation metric values, for the first inflammation metric, corresponding to a second inflammation tier, exceeding the first inflammation tier, of the first inflammation-related state; and
defining a third range of inflammation metric values, for the first
inflammation metric, corresponding to a third inflammation tier, exceeding the second inflammation tier, of the first inflammation-related state; and
wherein generating the inflammation-related state indication corresponding to the first inflammation-related state for the first patient comprises:
in response to the first inflammation metric value falling within the second range of inflammation metric values:
generating the inflammation-related state indication corresponding to the second inflammation tier of the first inflammation-related state for the first patient.

12. The method of claim 1:
wherein accessing the set of historical retinal scans comprises accessing the set of historical retinal scans, each historical retinal scan in the set of historical retinal scans tagged with patient demographic characteristics;
further comprising:
selecting a first subset of historical retinal scans, in the set of historical retinal scans, exhibiting a first set of patient demographic characteristics;
wherein generating the first inflammation function comprises generating the first inflammation function based on the first subset of historical retinal scans;
further comprising, during the training time period:
selecting a second subset of historical retinal scans, in the set of historical retinal scans, exhibiting a second set of patient demographic characteristics;
generating a second inflammation function, relating inflammation biomarkers with the first inflammation metric based on:
associations between constellations of inflammation biomarkers and a corresponding reference value of the first inflammation metric; and
the second subset of historical retinal scans;
associating the first inflammation function with the first set of patient demographic characteristics; and
associating the second inflammation function with the second set of patient demographic characteristics; and
further comprising, during the execution time period:
accessing a second set of patient demographic characteristics associated with the first patient; and
selecting the first inflammation function in response to the second set of patient demographic characteristics corresponding to the first set of patient demographic characteristics.

13. The method of claim 1:
wherein accessing the first retinal scan, captured by the retinal scanning system, for the first patient comprises accessing the first retinal scan of a first eye of the first patient;
further comprising, during the execution period:
accessing a second retinal scan, captured by the retinal scanning system, of a second eye of the first patient;
extracting a third constellation of inflammation biomarkers from the second retinal scan;
calculating a second inflammation metric value, for the first inflammation metric, based on the first inflammation function and the third constellation of inflammation biomarkers; and
calculating a bilateral similarity metric representing similarity between the first inflammation metric value and the second inflammation metric value; and
wherein generating the inflammation-related state indication corresponding to the first inflammation-related state for the first patient comprises:
in response to the bilateral similarity metric exceeding a threshold bilateral similarity metric and in response to the second inflammation metric value exceeding the threshold inflammation metric value:
generating the inflammation-related state indication corresponding to the first inflammation-related state for the
first patient.

14. A method comprising:
accessing a first retinal scan, captured by a retinal scanning system, for a first patient;
segmenting a first layer of the first retinal scan into a first set of segments of a first segment density;
for each segment in the first set of segments:
extracting a constellation of inflammation biomarkers from the segment;
calculating an intra-segment metric, in a first set of intra-tile metrics; representing spatial patterns between inflammation biomarkers in the constellation of inflammation biomarkers; and
calculating an inter-segment metric, in a first set of inter-segment metrics, representing relationships between the constellation of inflammation biomarkers and intra-segment metrics of segments in the first set of intra-segment metrics;
segmenting a second layer of the first retinal scan into a second set of segments of a second segment density less than the first segment density;
for each segment in the second set of segments:
extracting a constellation of inflammation biomarkers from the segment;
calculating an intra-segment metric, in a second set of intra-segment metrics, representing spatial patterns between inflammation biomarkers in the constellation of inflammation biomarkers; and
calculating an inter-segment metric, in a second set of inter-segment metrics, representing relationships between the constellation of inflammation biomarkers and intra-segment metrics of segment in the second set of intra-segment metrics;
calculating an inter-layer metric, representing systemic persistence of inflammation-related biomarker spatial patterns, based on:
the first set of intra-segment metrics;
the first set of inter-segment metrics;
the second set of intra-segment metrics; and
the second set of inter-segment metrics;
accessing a first inflammation function relating inflammation biomarkers with a first inflammation metric representing an inflammation-related state;
calculating a first inflammation metric value based on the inter-layer metric and the first inflammation function; and
in response to the first inflammation metric value exceeding a threshold inflammation metric value:
generating an inflammation-related state indication corresponding to the first inflammation-related state for the first patient; and
serving the first inflammation metric value and the inflammation-related state indication to an operator portal.

15. The method of claim 14:
wherein calculating the inter-segment metric, in the first set of inter-segment metrics, comprises:
for each segment in the first set of segments:
calculating a first variance score representing dispersion between the intra-segment metric of the segment and intra-segment metrics of segments in the first set of segments; and
assigning a first scale factor, in a first set of scale factors, to the segment inversely proportional to the first variance score; and
calculating the inter-segment metric, in the first set of inter-segment metrics, based on the first set of intra-segment metrics, each intra-segment metric in the first set of intra-segment metrics weighted by a corresponding scale factor in the first set of scale factors; and
wherein calculating the inter-segment metric, in the second set of inter-tile metrics, comprises:
for each segment in the second set of segments:
calculating a second variance score representing dispersion between the intra-segment metric of the segment and intra-segment metrics of segments in the second set of segments; and
assigning a second scale factor, in a second set of scale factors, to the segment inversely proportional to the second variance score; and
calculating the inter-segment metric, in the second set of inter-segment metrics, based on the second set of intra-segment metrics, each intra-segment metric in the second set of intra-segment metrics weighted by a corresponding scale factor in the second set of scale factors.

16. The method of claim 15:
further comprising calculating a confidence score inversely proportional to variance scores associated with segments in the first set of segments and the second set of segments; and
wherein generating the inflammation-related state indication corresponding to the first inflammation-related state for the first patient comprises:
generating the inflammation-related state indication corresponding to the first inflammation-related state for the first patient in response to the confidence score exceeding a threshold confidence score.

17. The method of claim 14:
further comprising accessing a set of regions associated with the first inflammation-related state;
for the first set of segments:
detecting a first subset of segments in a first region, corresponding to a region in the set of regions, of the first layer of the first retinal scan;
assigning a first scale factor to the first subset of segments;
detecting a second subset of segments in a second region, excluded from the set of regions, of the first layer of the first retinal scan; and
assigning a second scale factor, falling below the first scale factor, to the second subset of segment;
for the second set of segments:
detecting a third subset of segments in a third region, corresponding to a region in the set of regions, of the second layer of the first retinal scan;
assigning a third scale factor to the third subset of segments;
detecting a fourth subset of segments in a fourth region, excluded from the set of regions, of the second layer of the first retinal scan; and
assigning a fourth scale factor, falling below the third scale factor, to the fourth subset of segments; and
wherein generating the inflammation-related state indication corresponding to the first inflammation-related state for the first patient comprises generating a systemic state indication corresponding to the first inflammation-related state for the first patient.

18. The method of claim 14:
wherein accessing the first retinal scan, captured by the retinal scanning system, for the first patient comprises accessing the first retinal scan of a first eye of the first patient;
further comprising, during the execution period:
accessing a second retinal scan, captured by the retinal scanning system, of a second eye of the first patient;
extracting a third constellation of inflammation biomarkers from the second retinal scan;
calculating a second inflammation metric value, for the first inflammation metric, based on the first inflammation function and the third constellation of inflammation biomarkers; and
calculating a bilateral similarity metric representing similarity between the first inflammation metric value and the second
inflammation metric value; and
wherein generating the inflammation-related state indication corresponding to the first inflammation-related state for the first patient comprises:
in response to the bilateral similarity metric exceeding a threshold bilateral similarity metric and in response to the second inflammation metric value exceeding the threshold inflammation metric value:
generating the inflammation-related state indication corresponding to the first inflammation-related state for the first patient.

19. The method of claim 14:
wherein accessing the first retinal scan, captured by the retinal scanning system, for the first patient comprises accessing the first retinal scan, captured by the retinal scanning system during an execution time period, for the first patient; and
further comprising, during a training time period preceding the execution time period:
accessing a set of historical retinal scans, each historical retinal scan in the set of historical retinal scans tagged with a reference value of a first inflammation metric representing an inflammation-related state;
for each historical retinal scan in the set of historical retinal scans:
extracting a first constellation of inflammation biomarkers; and
associating the first constellation of inflammation biomarkers with the first inflammation metric; and
generating the first inflammation function relating inflammation biomarkers with the first inflammation metric based on associations between constellations of inflammation biomarkers and a corresponding reference value of the first inflammation metric.

20. A system comprising:
an optical sensor:
comprising:
an optical emitter configured to illuminate a retina of an eye of a patient; and
an optical detector configured to capture a retinal scan of the retina, illuminated by light emitted from the optical emitter;
an ocular interface configured to:
contact a brow of a patient; and
maintain a position of the optical sensor relative to the eye of the
patient during capture of the retinal scan; and
a controller configured to:
trigger capture of a first retinal scan, for a first patient, by the optical sensor;
extract a constellation of inflammation biomarkers from the first retinal scan;
calculate a first inflammation metric value, for the first inflammation metric, based on a first inflammation function and the constellation of inflammation biomarkers; and
in response to the first inflammation metric value exceeding a threshold inflammation metric value:
generate an inflammation-related state indication corresponding to the first inflammation-related state for the first patient; and
transmit the first inflammation metric value and the inflammation-related state indication to an operator portal.
